(19) United States
(12) Patent Application Publication
(10) Pub. No.: US 2010/0330049 A1
(43) Pub. Date: Dec. 30, 2010

(54) METHOD FOR GENERATING ISLET BETA CELLS FROM DEDIFFERENTIATED EXOCRINE PANCREATIC CELLS

(75) Inventors: Luc Bouwens, Dworp (BE); Luc Baeyens, Herzele (BE)

Correspondence Address: KNOBBE MARTENS OLSON & BEAR LLP, 2040 MAIN STREET, FOURTEENTH FLOOR, IRVINE, CA, 92614, US

(73) Assignees: Vrije Universiteit Brussel; Opus NV

(21) Appl. No.: 12/918,684

(22) PCT Filed: Feb. 11, 2009
(86) PCT No.: PCT/EP09/51542

§ 371 Date: Aug. 20, 2010

(30) Foreign Application Priority Data
Feb. 22, 2008 (EP) 08151856.5

(51) Int. Cl. A61K 35/39; C12N 5/02; C12N 5/071

(52) U.S. Cl. 424/93.7; 435/375; 435/325

(57) ABSTRACT

The present invention relates to an in vitro method for generating insulin-producing beta cells from a population of mammalian cells comprising dedifferentiated exocrine pancreatic cells. The method comprises the step of culturing said dedifferentiated exocrine pancreatic cells in a culture medium in the presence of at least one agent that is able to inhibit the Notch 1 signaling pathway in said dedifferentiated exocrine pancreatic cells, and at least one ligand of the gp130 receptor and/or at least one ligand of the EGF receptor. The invention further provides a population of mammalian pancreatic cells comprising insulin-producing beta cells obtainable by the present method and uses thereof in a pharmaceutical composition for treating type 1 or type 2 diabetes.

### FIELD OF THE INVENTION

The invention provides an in vitro method for generating insulin-producing beta cells from a population of mammalian cells comprising dedifferentiated exocrine pancreatic cells. The invention further provides a population of mammalian pancreatic cells comprising insulin-producing beta cells obtainable by the present method and a pharmaceutical composition comprising a pharmaceutical effective amount thereof. The present invention is further directed to a population of mammalian pancreatic cells or pharmaceutical composition as defined herein for use as a medicament or for use in treating type 1 or type 2 diabetes.

### BACKGROUND OF THE INVENTION

Precise regulation of circulating glucose levels, i.e., physiologically adequate glucose homeostasis, is essential for proper functioning and health of organisms. It is well-documented that disturbances of glucose homeostasis can hallmark and/or contribute to the aetiology of several prevalent diseases.

Insulin is a polypeptide hormone synthesised and secreted by the beta (β) cells of the Islets of Langerhans of the pancreas. The production of insulin in β cells responds to the presence and levels of circulating nutrients, in particular glucose. Insulin plays a central role in glucose homeostasis and causes a reduction in the circulating glucose levels, generally by increasing the uptake, metabolism and/or storage of glucose in cells of peripheral tissues, most prominently the adipose and muscle tissues. Accordingly, useful therapeutic interventions in disorders of glucose homeostasis may impinge on the endogenous insulin production, thereby advantageously increasing or decreasing the circulating glucose levels.

There are several conditions in which insulin disturbance is pathologic, including type I or type II Diabetes Mellitus.

Type I Diabetes Mellitus (T1DM) results from the autoimmune destruction of pancreatic beta cells. Type 1 diabetes is a hormone deficient state, in which the pancreatic beta cells appear to have been destroyed by the body's own immune defence mechanisms. The destruction of beta cells in type 1 diabetes leads to the inability to produce insulin, and thereby chronic insulin deficiency. Patients with type 1 diabetes have little or no endogenous insulin secretory capacity and develop extreme hyperglycemia. Type 1 diabetes was fatal until the introduction of insulin replacement therapy—first using insulins from animal sources, and more recently, using human insulin made by recombinant DNA technology.

Type 2 Diabetes Mellitus (T2DM) is typically a chronic, life-long disease characterized by insulin resistance. In clinical terms, insulin resistance is present when normal or elevated blood glucose levels persist in the face of normal or elevated levels of insulin. Symptoms may include excessive thirst, frequent urination, hunger, and fatigue. Hyperglycaemia associated with type 2 diabetes can sometimes be reversed or ameliorated by diet changes or weight loss which may at least partially restore the sensitivity of the peripheral tissues to insulin. Therapy in type 2 diabetes usually involves dietary therapy and lifestyle modifications. However, If after an adequate trial of diet and lifestyle modifications, fasting hyperglycemia persists, insulin therapy may be required to produce blood glucose control and, thereby, to minimize the complications of the disease. Progression of type 2 diabetes is associated with increasing hyperglycemia coupled with a relative decrease in the rate of glucose-induced insulin secretion. Therefore, for example, in late-stage type 2 diabetes there may be an insulin deficiency.

Replacement therapy of pancreatic beta cells under the form of islet cell transplantation is currently one of the best options for treatment of T1 DM, and can also be used to treat late-stage type 2 diabetes. It is however seriously hampered by the shortage of donor material, as well as the need for continuous immune suppression. Great effort is being put in looking for alternative sources of donor material like adult or embryonic stem cells, or expansion of pre-existing beta cells.

Methods for purifying insulin-producing cells that are present in the endocrine part of pancreas tissue have been disclosed, for instance in WO 03/093458. This document discloses a method for isolating regions, i.e. cells, capillaries or micro-organs, of interests from an organism based on the use of magnetic beads. An example of micro-organs includes islets of Langerhans.

With regard to the exocrine pancreas this can be a source of beta cell neogenesis. Specialized cells like those from the exocrine pancreas are generally considered to be the result of an unidirectional and irreversible process of differentiation under physiological conditions. Acinar cells display a remarkable plasticity and reportedly are able to convert their phenotype in vitro to duct, hepatocyte and beta cells.

For instance, Rooman et al. (2006 Amer. J. of Pathology, 169:4, p. 1206-1214) describe the acinoductal metaplasia, i.e. conversion of metaplastic (dedifferentiated) acinar cells into duct-like cells and indicates that Notch signaling is activated in acinar cells when they dedifferentiate into duct-like cells. This document does not refer to the redifferentiation of the dedifferentiated exocrine acinar pancreatic cells into beta cells. Redifferentiated duct-like cells are clearly different from and distinguishable from beta cells, since for instance only the latter contain insulin.

WO 2004/113512 discloses that, under appropriate culture conditions, Epidermal Growth Factor (EGF) and Leukemia Inhibitory Factor (LIF) can induce dedifferentiated acinar cells to partially recapitulate the beta cell embryonic ontogeny. Hereby cells become re-specified to insulin-expressing cells via transient expression of the pro-endocrine transcription factor Neurogenin-3 (Ngn3). During embryonic pancreas development, Ngn3 is transiently expressed by the precursors of endocrine cells, and introduction of exogenous Ngn3 in adult duct cells can initiate pro-endocrine differentiation. However, the process disclosed in WO 2004/113512 has the disadvantage of having a low efficiency and resulting in a low number of acinar cells having adopted a beta cell phenotype.

There is therefore a need in the art for other methods for the preparation of islet beta cells from exocrine pancreatic cells which overcome at least some of the above cited disadvantages or problems of prior art methods.

### SUMMARY OF THE INVENTION

It is an object of the present invention to provide an improved method for the neogenesis of beta cells. More in particular, it is an object of the invention to provide a method for the generation of new insulin-producing cells from insulin-negative precursor cells in the exocrine part of the pancreas, and more in particular to a method for the generation of insulin-producing beta cells from insulin-negative exocrine pancreatic cells.

The present invention provides in general a method for generating islet beta-cells from exocrine pancreas cells. The present invention underscores the principle and potential application of reprogramming a mature differentiated cell type by physiological signals into a different mature cell type. In particular, the present invention provides a method by which the efficiency of generating islet beta-cells from dedifferentiated exocrine pancreas cells is greatly improved and more insulin-positive cells can be obtained compared to available prior art methods. The pre-sent invention is at least in part based on the Aplicant's finding that by inhibiting the Notch signaling pathway in dedifferentiated exocrine pancreatic cells, an efficient model is obtained for the generation of beta cells suitable for restoration of normoglycemia in diabetic animals, mammals, and in particular humans.

In a first aspect, the invention is therefore directed to an in vitro method of generating insulin-producing beta cells from a population of mammalian cells comprising dedifferentiated exocrine pancreatic cells comprising the step of culturing said dedifferentiated exocrine pancreatic cells in a culture medium in the presence of
at least one agent that is able to inhibit the Notch1 signaling pathway in said dedifferentiated exocrine pancreatic cells, and
at least one ligand of the gp130 receptor and/or at least one ligand of the EGF receptor.

In one embodiment, a method is provided wherein said dedifferentiated exocrine pancreatic cells are cultured in a culture medium in the presence of at least one agent that is able to inhibit the Notch1 signaling pathway in said dedifferentiated exocrine pancreatic cells, at least one ligand of the gp130 receptor, and at least one ligand of the EGF receptor.

In accordance with the present invention a cell culturing method was developed to convert pancreatic cells, and preferably acinar cells, into endocrine beta cells based on reprogramming dedifferentiated pancreatic cells and preferably dedifferentiated acinar cells, with agent(s) A) able to inhibit Notch1 signaling pathway, and B) ligand(s) of the gp130 receptor and/or ligand(s) of the EGF receptor, e.g. LIF and EGF respectively. The applicant has shown that physiological or RNAi-based interference with the Notch1 signaling pathway allows modulating the susceptibility of dedifferentiated pancreatic cells to the differentiation-inducing factors and significantly improves beta cell neoformation. The applicant further showed that newly formed beta cells further mature when transplanted ectopically, and are capable of restoring normal blood glycemia in diabetic recipients.

In a preferred embodiment, the present invention provides a culturing method wherein the agent that is able to inhibit the Notch1 signaling pathway is an agent able to reduce the expression of Notch1 or Hes gene(s), preferably Hes1, and preferably is an agent capable of causing RNA interference with Notch1 or a Hes gene, preferably Hes1.

More preferably, the present invention provides a culturing method wherein said agent capable of causing RNA interference with Notch1 or a Hes gene, preferably Hes1, is a RNA interfering agent chosen from the group comprising short interfering nucleic acid (siNA), short interfering RNA (sRNA), double-stranded RNA (dsRNA), micro-RNA (miRNA), and short hairpin RNA (shRNA). Preferably, said RNA interfering agent is produced by chemical synthesis, by enzymatic synthesis or recombinantly expressed from a vector in a cell.

In another embodiment the invention provides a culturing method wherein the agent that is able to inhibit the Notch1 signaling pathway is a Notch1-inhibiting agent. According to one embodiment said Notch1-inhibiting agent is a human or humanised Notch1-inhibiting agent. According to a preferred embodiment said Notch1-inhibiting agent is Notch1-EC, such as human or humanised Notch1-EC. According to another embodiment, Notch1-EC is added to the culture medium in a concentration between 1 and 70 μg/ml, preferably between 3 and 60 μg/ml or between 5 and 50 ng/ml.

According to another embodiment the ligand of said gp130 receptor is a human or humanised ligand of said gp130 receptor. According to another embodiment, the ligand of said gp130 receptor is LIF, such as human or humanised LIF. According to one embodiment LIF is added to the culture medium in a concentration between 10 and 100 ng/ml, or between 10 and 25 ng/ml or between 100 and 500 ng/ml.

According to yet another embodiment the ligand of said EGF receptor is a human or humanised ligand of said EGF receptor. According to another embodiment the ligand of said EGF receptor is EGF such as human or humanised EGF.

According to one embodiment LIF is added to the culture medium in a concentration between 10 and 100 ng/ml, or between 10 and 25 ng/ml or between 100 and 500 ng/ml.

The invention provides a method wherein the mammalian cells can be rodent, porcine, monkey and human cells, and preferably are human cells.

Advantageously, the present invention provides a method wherein more than 20%, preferably more than 25%, preferably more than 30%, preferably more than 35%, preferably more than 40%, preferably more than 50% of dedifferentiated exocrine pancreatic cells adopt a beta cell phenotype.

The applicant showed that a method wherein dedifferentiated exocrine pancreatic cells are cultured in a culture medium comprising A) at least one agent that is able to inhibit the Notch1 signaling pathway in said dedifferentiated exocrine pancreatic cells, and B) at least one ligand of the gp130 receptor and/or at least one ligand of the EGF receptor, has a significantly higher efficiency than if the cells would be cultured in a culture medium comprising at least one ligand of the gp130 receptor and/or at least one ligand of the EGF receptor. Whereas the latter methods induce the dedifferentiation of less than 10% pancreatic (e.g. acinar) cells into beta cells, the present method induces the dedifferentiation of more than 20% pancreatic (acinar) cells into beta cells, i.e. more than a two-fold increase.

It is further an object of the invention to provide a population of mammalian pancreatic cells comprising insulin-producing beta cells that have been obtained from dedifferentiated exocrine pancreatic cells. In particular, the invention provides a population of mammalian pancreatic cells comprising insulin-producing beta cells that have been obtained from dedifferentiated exocrine acinar cells.

Another aspect of the invention relates to a population of mammalian pancreatic cells comprising mammalian insulin-producing beta cells obtained or obtainable by any of the herein described embodiments of the method of the present invention.

As indicated above, application of islet transplantation as a treatment for diabetes is hampered by an inadequate supply of insulin-producing cells. In the present invention insulin-producing beta cells are generated from exocrine pancreatic cells, which represent the great majority of cells in the pancreas, e. g. in humans and other mammals. The present invention provides a method wherein beta-cell neogenesis is induced from exocrine cells by culturing the cells in the presence of two soluble factors provided in the culture medium, namely EGF and LIF, and in combination therewith by inhibiting Notch1 signaling pathway in these cells, either using a notch1-inhibiting agents or a RNAi based approach for reducing the expression of Notch1 and/or primary target genes thereof such as Hes genes, preferably Hes1. The invention provides an important advancement in the treatment of diabetes by islet transplantation, by providing a way to overcome the problem of insufficient donor beta cells. The ability to generate new functional beta cells in vitro could alleviate the need for insulin substitution and benefit patient care.

Therefore in another aspect, the invention provides a pharmaceutical composition comprising a therapeutically effective amount of a population of mammalian pancreatic cells as defined herein and at least one pharmaceutically acceptable carrier.

In another embodiment, the invention further provides a population of mammalian pancreatic cells as defined herein for use as a medicament.

In yet another embodiment the invention provides a population of mammalian pancreatic cells as defined herein for treating type 1 or type 2 diabetes. The invention is also directed to the use of a population of mammalian pancreatic cells as defined herein for the preparation of a medicament for treating type 1 or type 2 diabetes.

The invention further provides a method of treating type 1 or type 2 diabetes comprising administering to a subject tin need thereof a therapeutically effective amount of a population of mammalian pancreatic cells as defined herein or a pharmaceutical composition as defined herein.

In still another aspect, the present invention is also directed to a method of cell tracing based on specific incorporation of lectin to demonstrate the conversion of dedifferentiated exocrine pancreatic cells to beta cells. In another aspect, the invention therefore provides a method for tracing the origin of insulin-producing beta cells obtained from dedifferentiated pancreatic cells according to the present method comprising the steps of:
a) providing a population of dedifferentiated exocrine pancreatic cells in a culture medium,
b) labeling said dedifferentiated exocrine pancreatic cells, preferably with a fluorescent label, preferably fluorescent lectin, more preferably fluorescent wheat germ agglutinin,
c) culturing the labeled dedifferentiated exocrine pancreatic cells according to a method as defined herein thereby obtaining insulin-producing beta cells, and
d) determining the presence of the fluorescent label in said insulin-producing beta cells.

In vitro labeling is exocrine specific; this labeling method enables to label all exocrine cells but no endocrine cells.

### DETAILED DESCRIPTION OF THE FIGURES

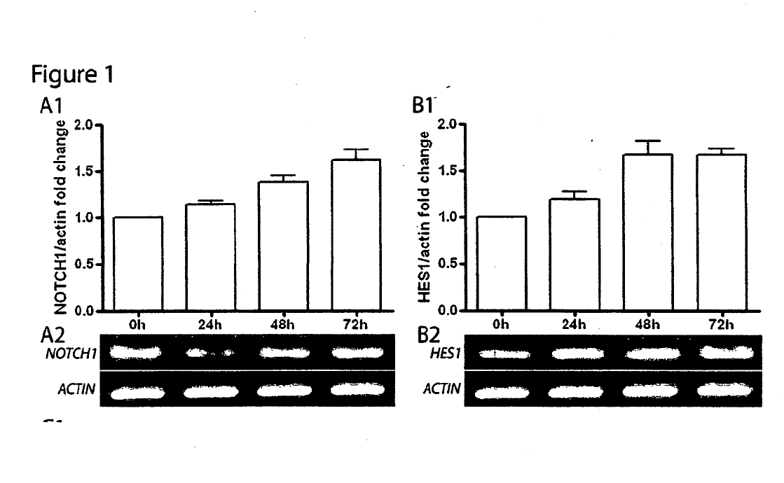

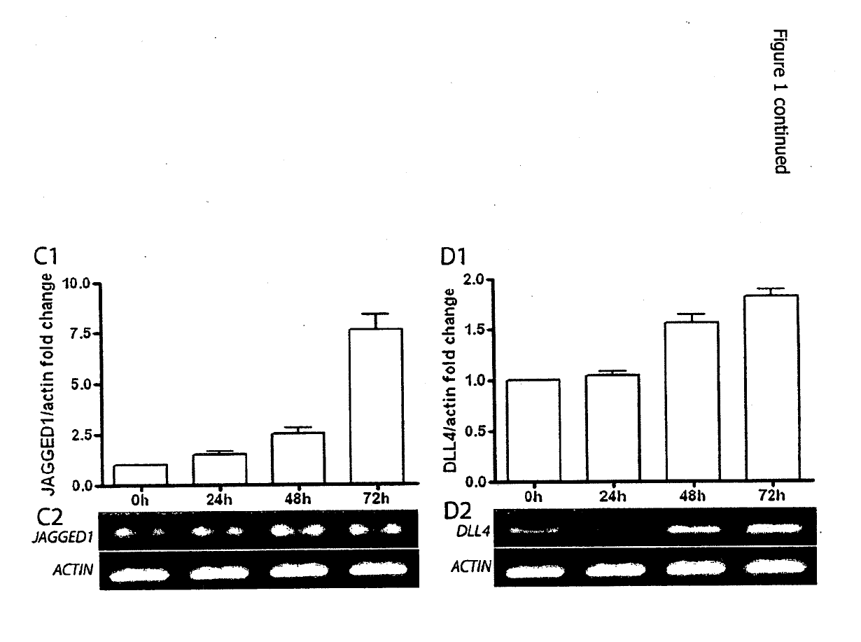

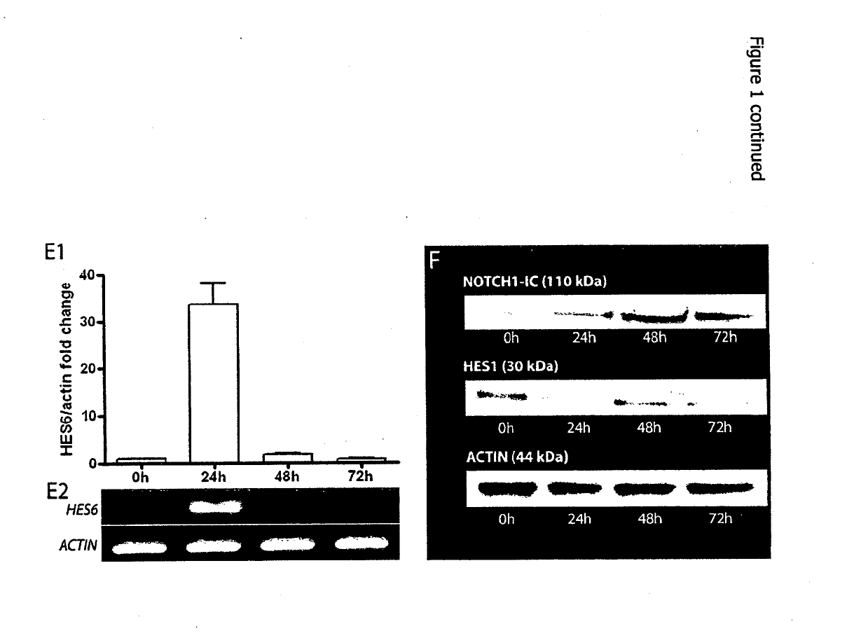

FIG. 1 illustrates the Notch pathway expression profile in EGF/LIF treated acinar cells. (A-E) Gene expression of Notch1 pathway related genes in EGF/LIF treated acinar cells. Both quantitative (A1-E1) and conventional (A2-E2) RT-PCR data are presented. All qPCR data are corrected for β-actin expression and normalized to the control condition of 0 h of treatment with EGF and LIF. (F) Protein expression pattern of activated Notch1 (Notch1-IC) and the main effector Hes1 in EGF and LIF treated cultures over time. Actin is used as internal loading control. Error bars represent mean±s.e.m. (n=4).

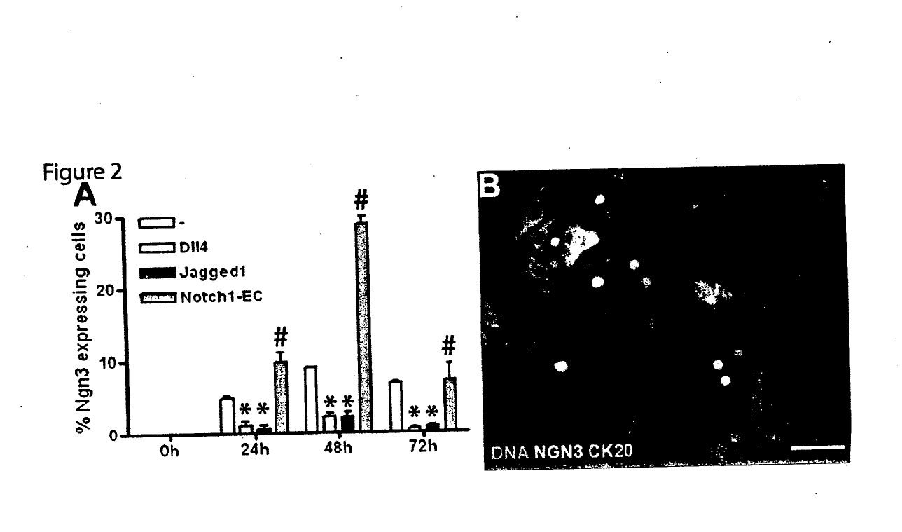

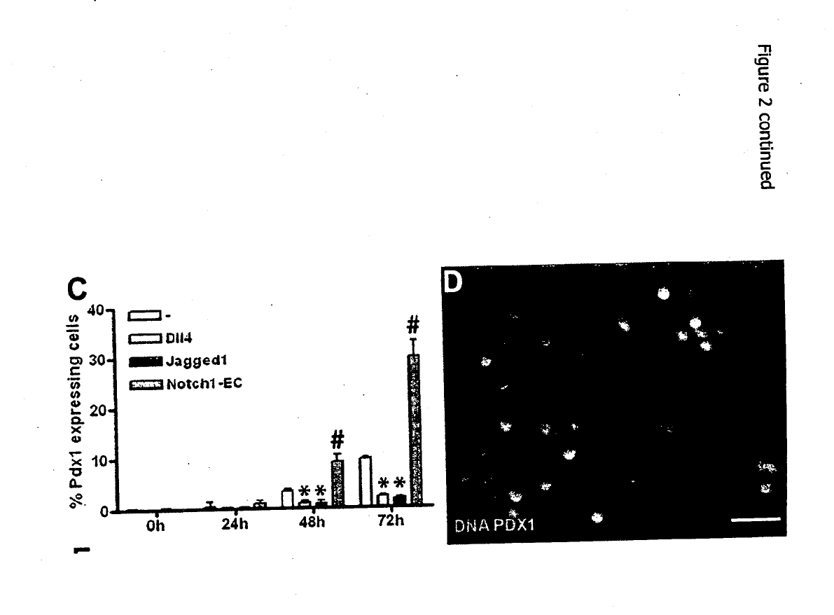

FIG. 2 illustrates that exogenous Notch1-EC, Jagged1 and Dll4 modulate beta cell conversion of mature acinar cells. Error bars represent mean±s.e.m. Scale bar=15 μm. (A and B) During the 72 h exposure to differentiation factors EGF and LIF, acinar cells (white bars) exhibit a limited and temporal re-expression of the pro-endocrine transcription factor Ngn3. Addition of exogenous ligands Jagged1 (▪) or Dll4 [symbol] almost completely abrogates this pattern, whereas adding Notch1-EC (extracellular domain of the receptor) [symbol] enhances the potency of the differentiation factors (n=7; *p=[0.022-0.031]; #p=[0.007-0.019] compared to controls). Panel B shows Ngn3-positive cells (green) in a culture of dedifferentiated acinar cells (characterized by cytokeratin 20 expression (red)) 48 h after initiation of treatment with EGF, LIF and Notch1-EC. (C and D) During the 72 h exposure to differentiation factors EGF and LIF, acinar cells (white bars) start to express the beta cell transcription factor Pdx1 towards the end of the treatment (n=7). Addition of exogenous ligands Jagged1 (▪) or Dll4 [symbol] prevents this expression, whereas adding Notch1-EC [symbol] amplifies the number of cells expressing Pdx1 (n=7; *p=[0.032-0.043]; #p=[0.011-0.037] compared to controls). Panel D shows Pdx1-positive cells (red) in the culture 72 h after initiation of treatment with EGF, LIF and Notch1-EC. (E and F) During the 72 h exposure to differentiation factors EGF and LIF, a part of the acinar cells (white bars) start to express insulin towards the end of the treatment. Addition of exogenous ligands Jagged1 (▪) or Dll4 [symbol] prevents this expression, whereas adding Notch1-EC [symbol] amplifies the number of insulin-positive cells in these conditions (n=7; *p=[0.023-0.029]; #p=[0.009-0.038] compared to controls). Panel F shows insulin-positive cells (green) in the culture 72 h after initiation of treatment with EGF, LIF and Notch1-EC.

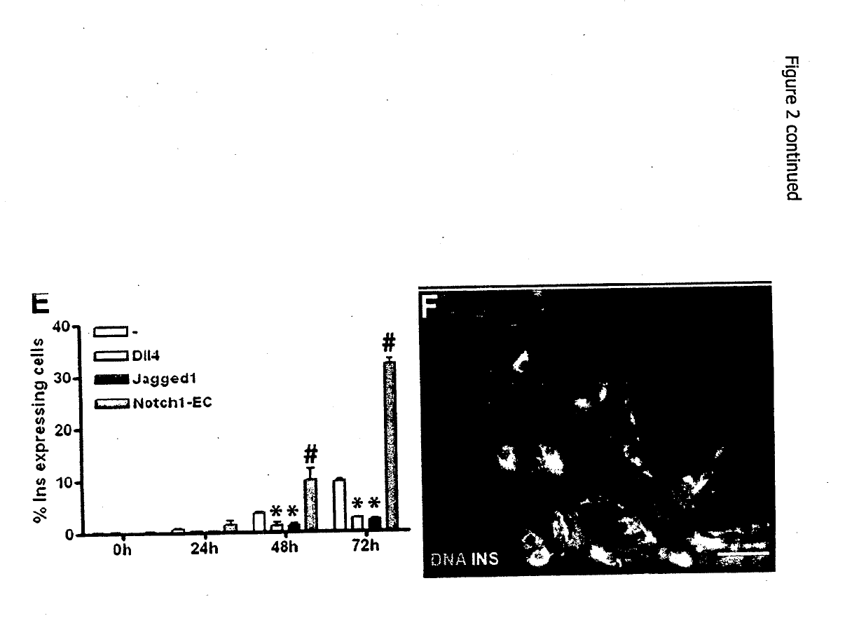

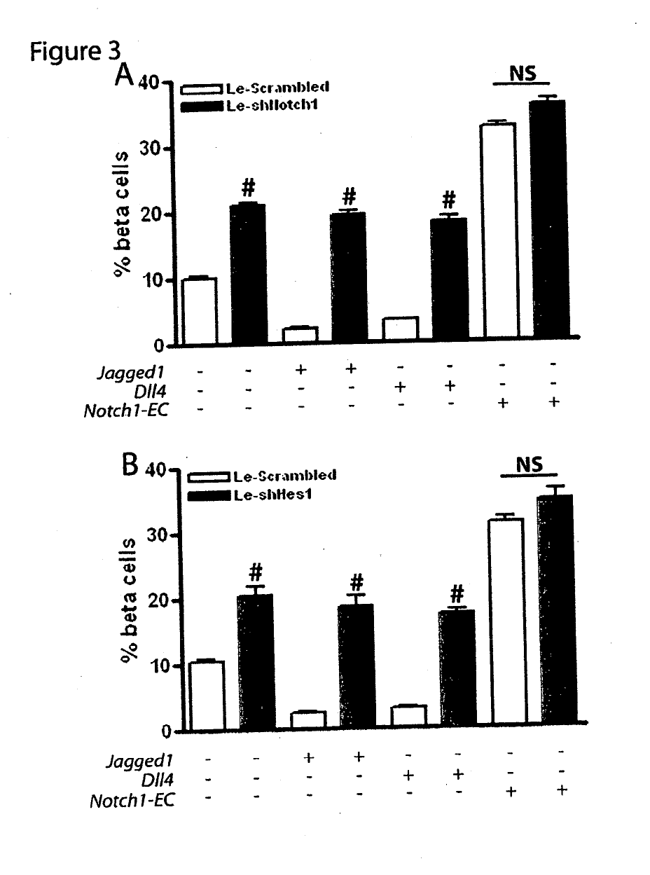

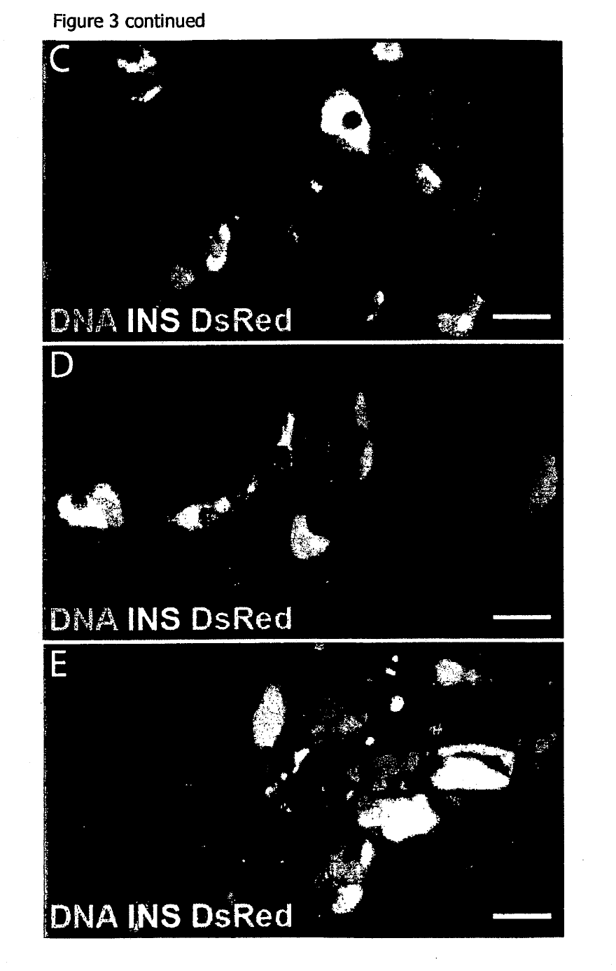

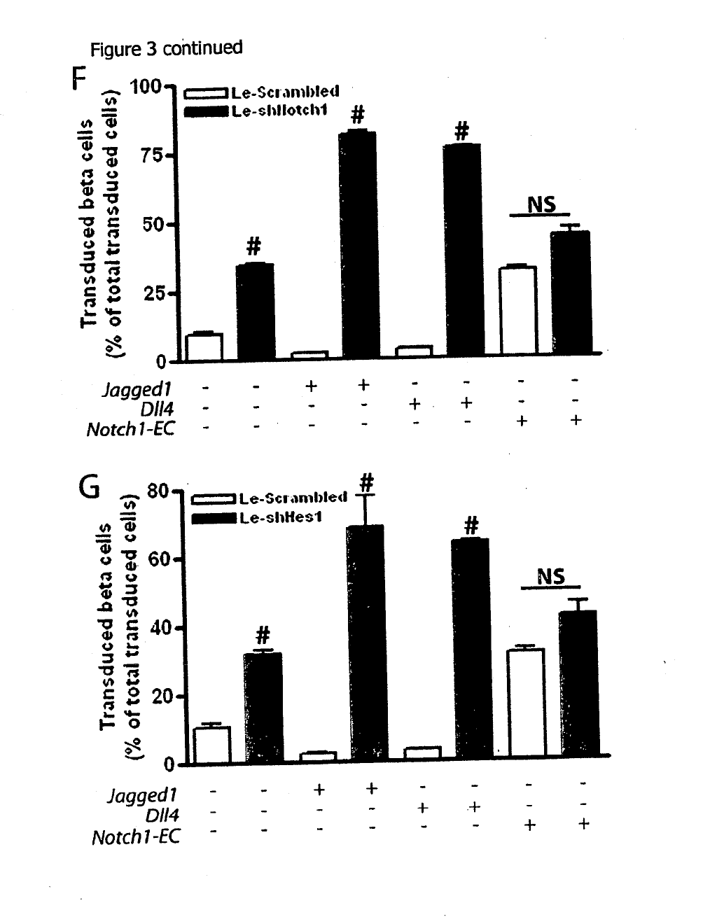

FIG. 3 illustrates that RNA interference of Notch signaling counteracts Jagged1 and Dll4 inhibition of acinar-to-beta cell conversion. Error bars represent mean±s.e.m. (A and B) Effect of lentiviral introduction of shRNA directed against either Notch1 (A) or Hes1 (B) on the proportion of beta cells. Transduction of the cells with shNotch1 or shHes1 induced a significant increase in the proportion of beta cells (n=4; resp. #p=0.039 and #p=0.041 compared to shScrambled condition). Additional treatment with the ligands Jagged1 or Dll4 emphasized the effect of the specific knockdown (shNotch1 or shHes1) on the beta cell differentiation (n=4; resp. #p=0.009 and #p=0.011 compared to shScrambled condition) as very little beta cells are present in the condition with non-specific knockdown. In conditions treated with Notch1-EC, displaying an enhanced beta cell differentiation, no apparent effect of additional interference of Notch1 or Hes1 was observed (n=4; resp. p=0.12 and p=0.14). (C-E) Illustration of co-expression of the beta cell marker insulin and DsRed reporter protein in conditions transduced with Le-shNotch1-DsRed (C), Le-shHes1-DsRed (D) or Le-Scrambled-DsRed (E) and treated with the differentiation factors. More insulin/reporter co-expression was observed after specific silencing of the Notch pathway (C and D) compared to controls (E). Scale bar=10 μm. (F and G) Co-expression of the beta cell marker insulin with the reporter is a measure for the susceptibility of the cells to the pro-endocrine treatment after Notch pathway silencing. Transduction of the cells with shNotch1 (F) or shHes1 (G) led to a significant increase in transduced beta cells (n=4; resp. #p=0.035 and #p=0.037 compared to shScrambled condition). Exposure to the ligands Jagged1 or Dll4 reduced the proportion of shScrambled transduced beta cells severely, whereas a clear protective effect of specific Notch pathway silencing (shNotch1 or shHes1) against the action of Notch ligands was demonstrated by the high amount of silenced cells adopting a beta cell phenotype (n=4; resp. #p=0.005 and #p=0.009 compared to shScrambled condition). In conditions treated with Notch1-EC, no difference in the proportion of transduced beta cells after interference with Notch1 or Hes1 was observed when compared to control shRNA (n=4; resp. p=0.061 and p=0.074). (H-J) Evaluation of the role of Ngn3 in conditions treated with EGF, LIF and Notch1-EC. The silencing of Ngn3 completely abrogates the co-expression of the reporter eGFP with Ngn3 protein compared to controls (H) (n=3; #p=0.007). Immunocytochemical analysis revealed the presence of Ngn3/reporter co-expressing cells after 48 h of pro-endocrine treatment in the shScrambled condition (I), contrasted by the complete absence of these cells after specific Ngn3 silencing (J).

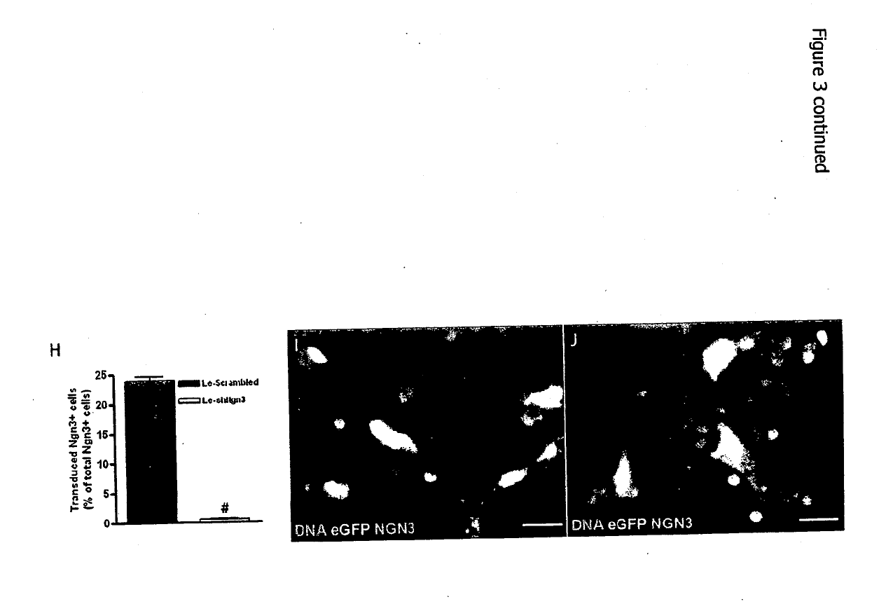

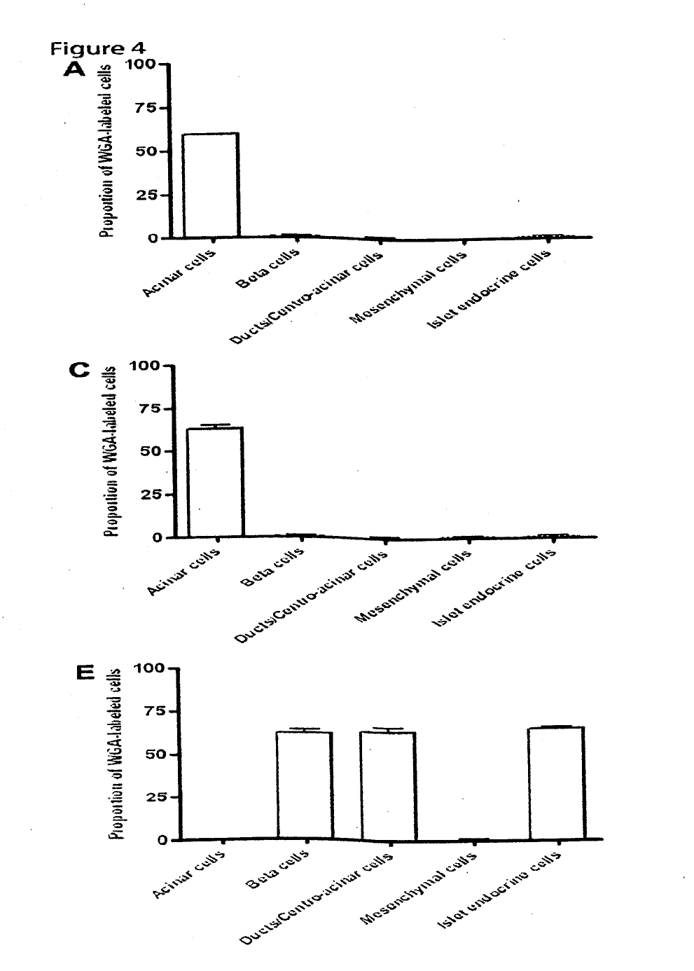

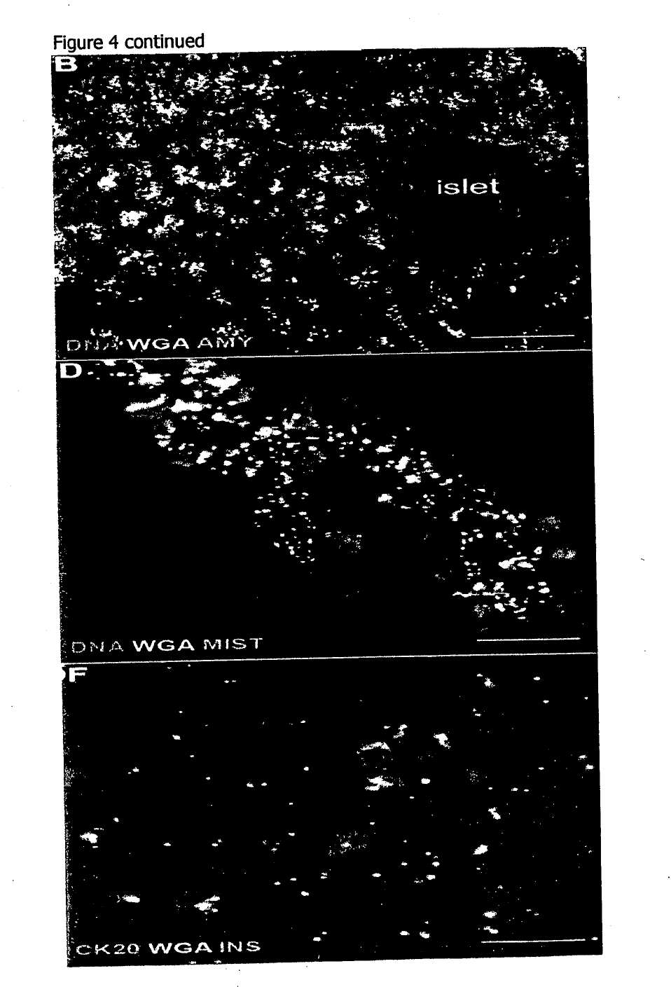

FIG. 4 shows that newly generated beta cells are of acinar origin. Error bars represent mean±s.e.m. (A-B) WGA label specificity and efficiency in the pancreas 72 h after intraparenchymal micro-injection of the lectin. WGA labels specifically acinar cells with an efficiency of 59.7±0.3% (n=4) and is not found in any other pancreatic cell type (A). Immunostaining for amylase (red) reveals co-expression with the lectin label (green) (B). Scale bar=100 μm. (C-D) WGA label specificity and efficiency evaluated after isolation of the pancreatic acini. WGA label was observed in 62.8±2.2% of the acinar cells (n=4), and was absent in other pancreatic cell types (C). Staining for the acinar transcription factor Mist1 (red) shows that the WGA lectin (green) is only present in acinar cells (D). Scale bar=15 μm. (E-F) WGA label specificity and efficiency evaluated after suspension culture (96 h) to induce acinar dedifferentiation, followed by the beta cell differentiation treatment on monolayer cultures (72 h). Cells have lost their acinar phenotype and gained a duct-like morphology (characterized by expression of cytokeratin 20). 63.5±2.3% (n=4) of the cytokeratin-positive fraction contained the lectin label and, after pro-endocrine treatment, 62.1±1.7% (n=4) of the beta cells contained WGA (E). Staining for the ductal marker cytokeratin 20 (blue) and beta cell marker insulin (red) showed a co-expression with the acinar cell tracer WGA (green) (F). Scale bar=15 μm. These observations demonstrate that the in vivo labeled acinar cells converted into beta cells and ductal cells after in vitro treatment.

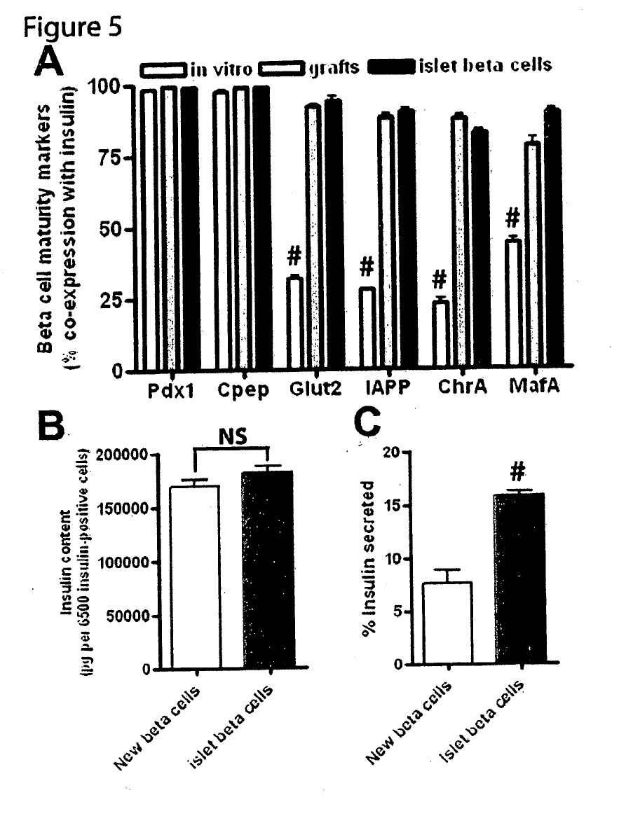

FIG. 5 shows in vitro maturity profile of newly formed beta cells. Error bars represent mean±s.e.m. (A) Analysis of phenotypical maturity of the new beta cells by immunocytochemistry. New beta cells (□) express Pdx1 and C-peptide at the same level as islet beta cells (▪) (n=4; p=0.32), but a significantly lower proportion of the beta cells expressed other maturity markers such as Glut2, IAPP, ChromograninA and MafA (n=4; #p=[0.006-0.018] compared to islet beta cells). After transplantation of the cells at an ectopic site in diabetic nude mice the grafted beta cells [symbol] matured to the level of islet beta cells (n=4; p=[0.45-0.57]). (B) Comparison of the cellular insulin content of new beta cells (white bars) and islet beta cells (red bars). No significant difference between both groups was detected (n=3; p=0.28). (C) Percentage of total cellular insulin secreted after stimulation with 20 mM glucose from new beta cells (white bars) and islet beta cells (red bars); a limited capacity for glucose-induced insulin secretion is seen in new beta cells as compared to mature beta cells (n=3; #p=0.023).

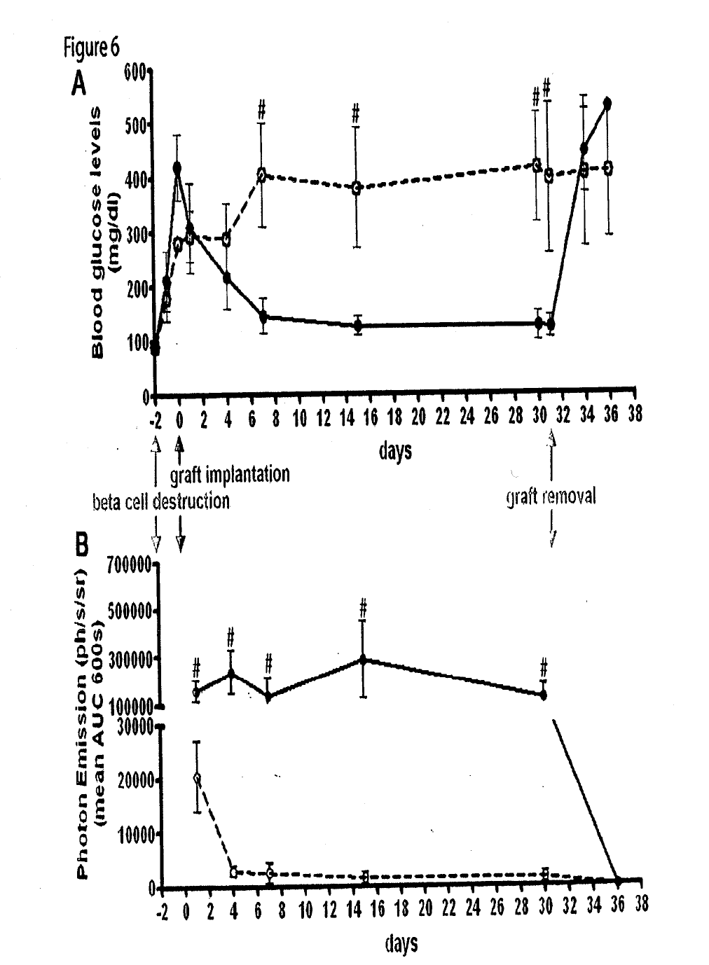

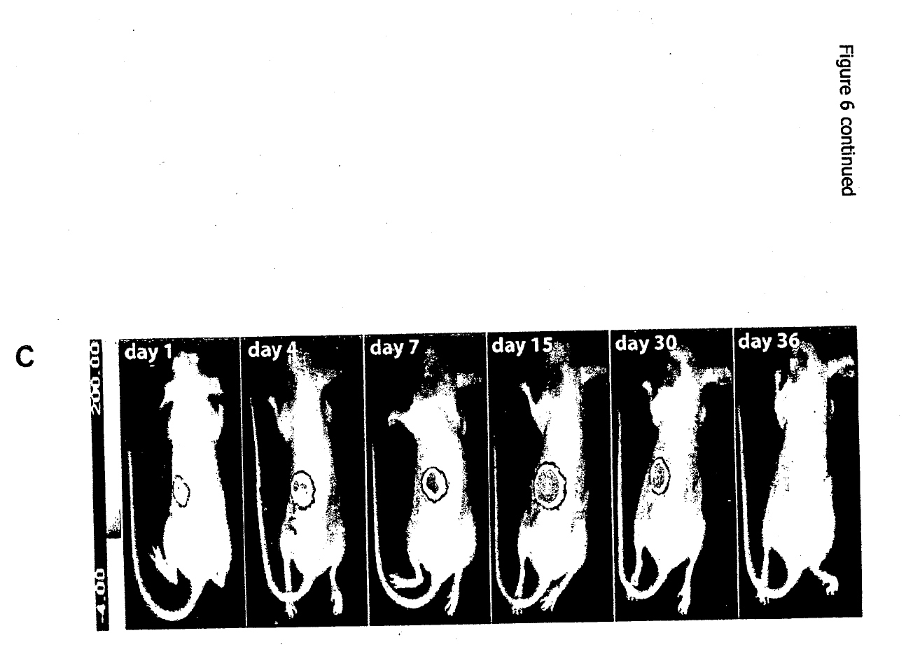

FIG. 6 shows that beta cells from acinar origin are capable to revert hyperglycaemia upon transplantation in diabetic animals. (A) Immunodeficient mice were injected with alloxan at day -2 to induce total beta cell destruction and subsequent hyperglycemia. Upon transplantation of acinar-derived beta cells pre-treated with EGF, LIF and Notch1-EC (−), under the kidney capsule on day 0 (1×10E5 new beta cells per animal), normoglycemia was restored (indicated by yellow area). Nephrectomy of the graft-bearing kidney on day 31 resulted in acute reversal to the diabetic state, proving that the grafted cells were able to cure diabetes. Mice transplanted with control grafts (no pro-endocrine treatment in vitro) remained hyperglycemic at all time points (−) (n=4; #p=[0.037-0.047]). (B) Lentivirus-introduced Luciferase-labeling of the transplanted cells allowed for non-invasive imaging and revealed the formation of grafts with a stable mass when they had been pre-treated with EGF, LIF and Notch1-EC to induce acinar-to-beta cell conversion (−). Control grafts displayed immediate deterioration in the intensity of the luminescent signal. By day 4, the signal was almost completely abrogated, indicative for loss of the graft (−) (n=4; #p=[0.008-0.031]). (C) Visualization of the luminescent signal in a mouse engrafted with EGF, LIF and Notch1-EC pre-treated cells. Luminescent signals correspond to the ectopic site of implantation and display stable signal intensity until the graft was surgically removed.

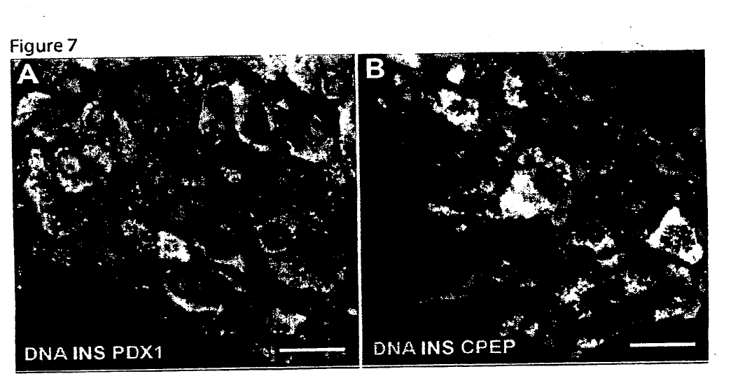

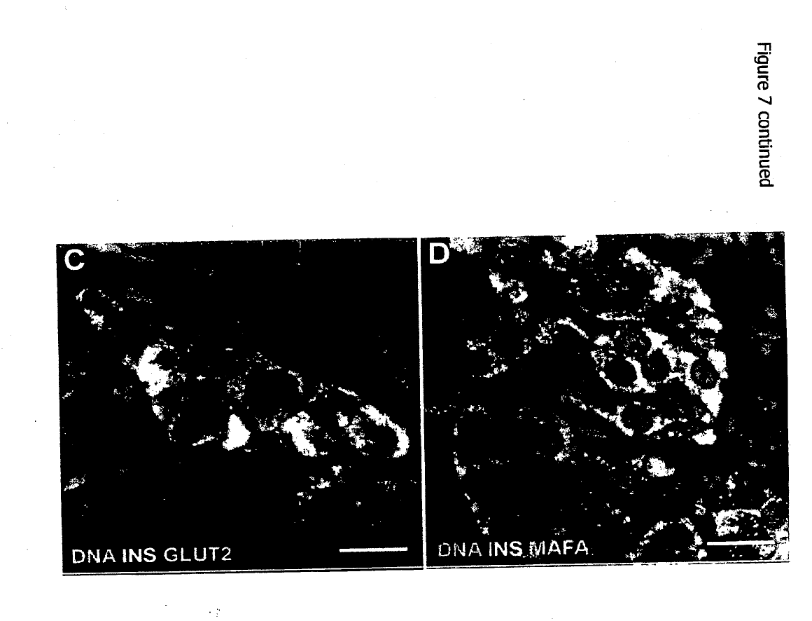

FIG. 7 illustrates phenotypical analysis of the grafted cells. (A-D) Upon removal of the graft, the maturity of the beta cells was evaluated by immunohistochemistry. Double staining using anti-insulin (green) and anti-Pdx1 (red) (A), anti-C-peptide (red) (B), anti-Glut2 (red) (C) or anti-MafA (red) (D) demonstrated that almost all cells co-expressed both markers. In contrast to their in vitro phenotype, the acinar-derived beta cells underwent an in vivo maturation process post-implantation. Scale bar=10 μm. (E) Staining using anti-Luciferase (green) confirmed Luciferase expression in about 30% of the insulin-positive cells (red). Scale bar=10 μm. (F) Histological analysis of control pancreas (F1) or pancreas from alloxan-injected, engrafted mice (F2). In contrast to normal islet distribution in control pancreas, insulin-positive cells were nearly totally absent after alloxan-mediated beta cell destruction. Scale bar=500 μm.

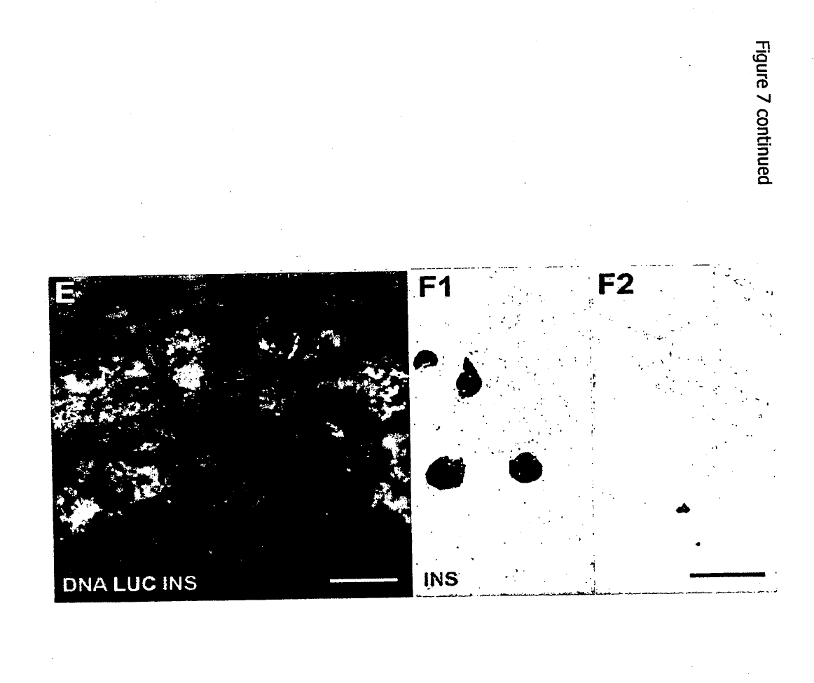

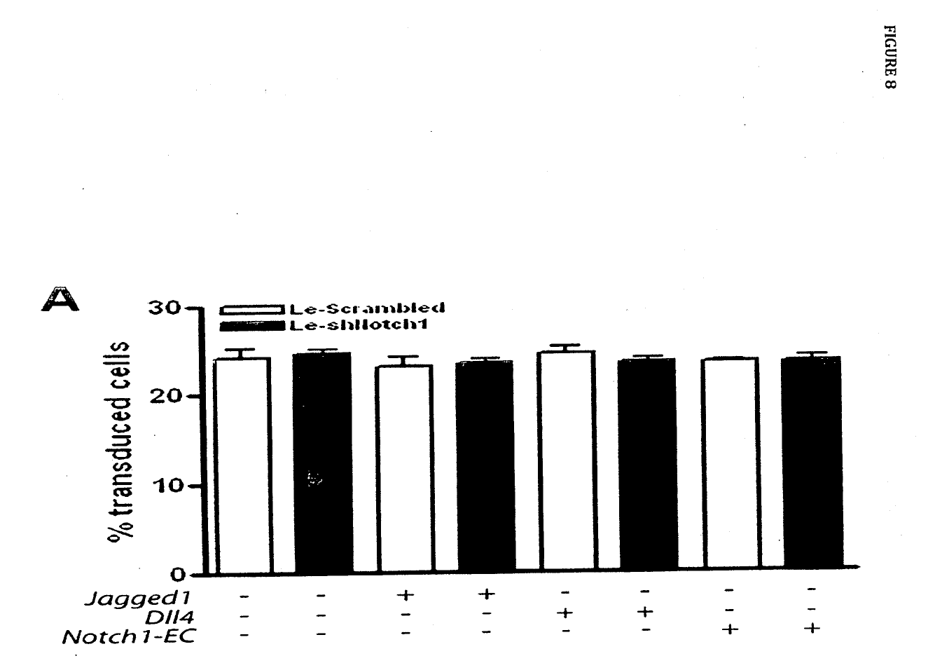

FIG. 8 shows transduction efficiency of the different silencing viruses. (A and B) Proportion of cells transduced after infection with lentivirus containing shRNA directed against either Notch1 (A) or Hes1 (B). No significant differences in transduction efficiency was noted between the different viruses (n=3; p>0.05 compared to shScrambled condition). Additional treatment with the ligands Jagged1 or Dll4 or with the extracellular part of the Notch1 receptor (Notch1-EC) induced no difference in the proportion of transduced cells (n=3; p>0.05). (C) Evaluation of the transduction efficiency after infection with lentiviruses Le-shScrambled-eGFP and Le-shNgn3-eGFP in conditions treated with EGF, LIF and Notch1-EC. No significant differences between both conditions were observed (n=3; p>0.05).

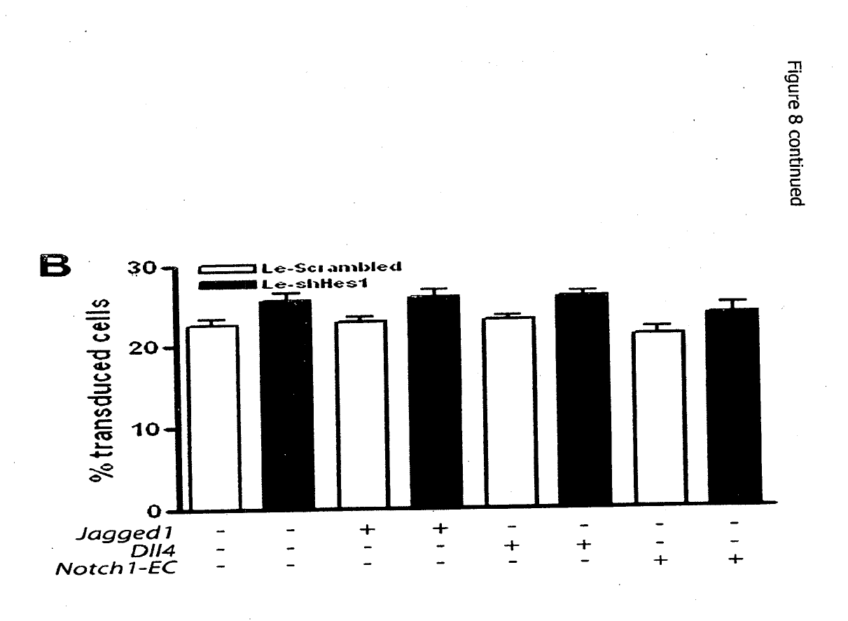

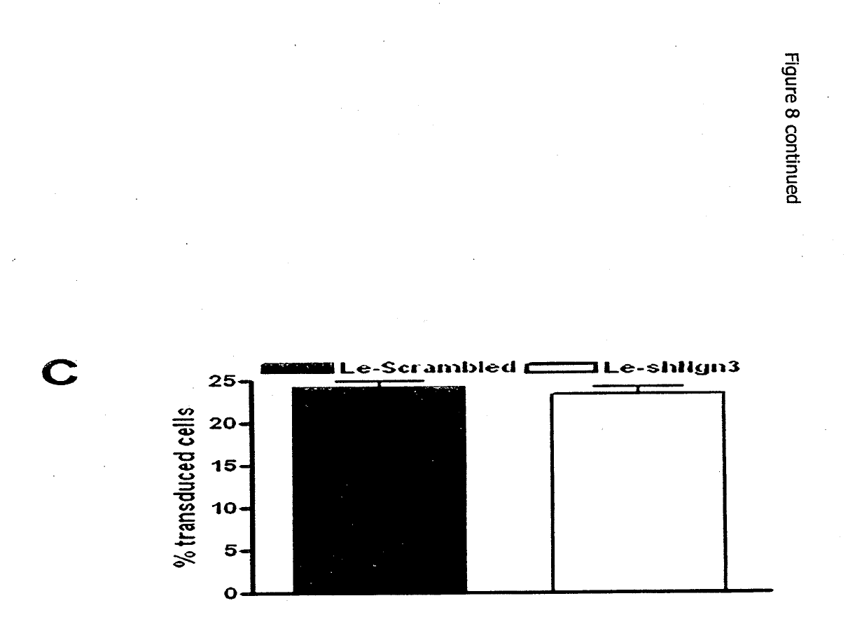

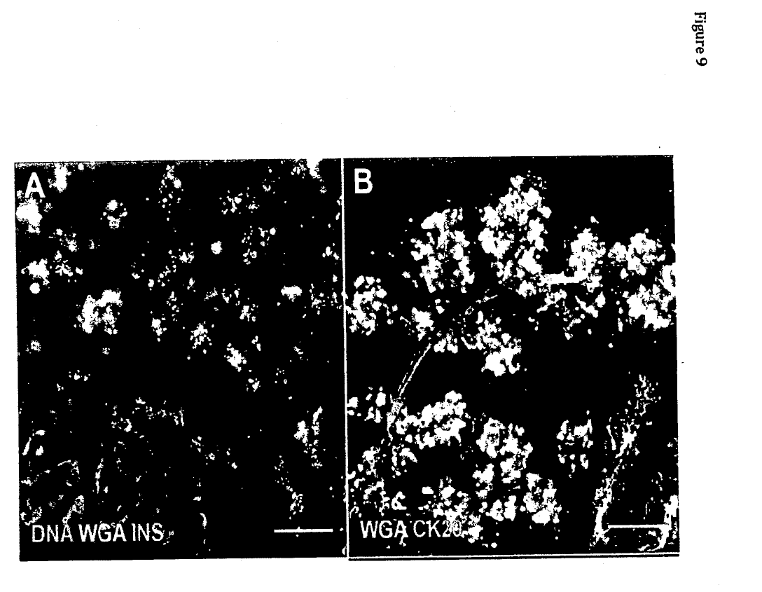

FIG. 9 shows wheat Germ Agglutinin specificity after injection and isolation. (A) WGA label specificity and efficiency in the pancreas 72 h after intraparenchymal micro-injection of the lectin. Co-localization of the fluorescent lectin with beta cells was never observed in this condition (n=4). Immunostaining for insulin (red) reveals the expression of the lectin label (green) is separated from the beta cell population. Scale bar=100 μm. (B) WGA label specificity and efficiency evaluated after partial dissociation and isolation of the pancreatic acini. WGA label was observed in the acinar cells, but not in ductal or centro-acinar cells. Staining for the duct/centro-acinar phenotypical marker cytokeratin 20 (CK20) (red) indicated that the WGA lectin (green) was only present in acinar cells. Scale bar=25 μm. (C) After dissociation of the pancreas, pancreatic islets were isolated, dissociated and the beta cells were purified using FACS. Fluorescence-activated cell sorting revealed a subpopulation (2.4% of the total cell population) that displayed lectin positivity based on fluorescein intensity. (D) Immunocytochemical analysis of this subpopulation using staining for the beta cell marker insulin (red) showed no co-expression of beta cells with the acinar cell tracer WGA (green). Scale bar=15 μm.

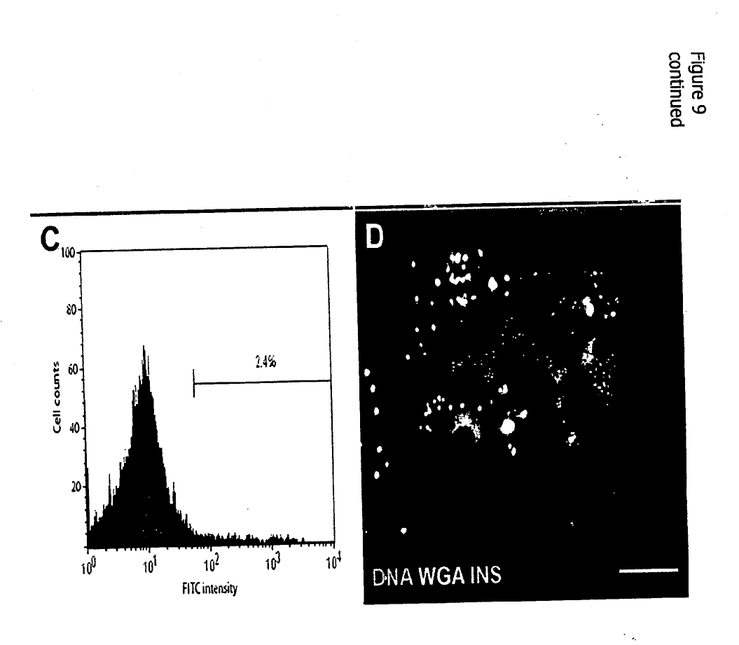

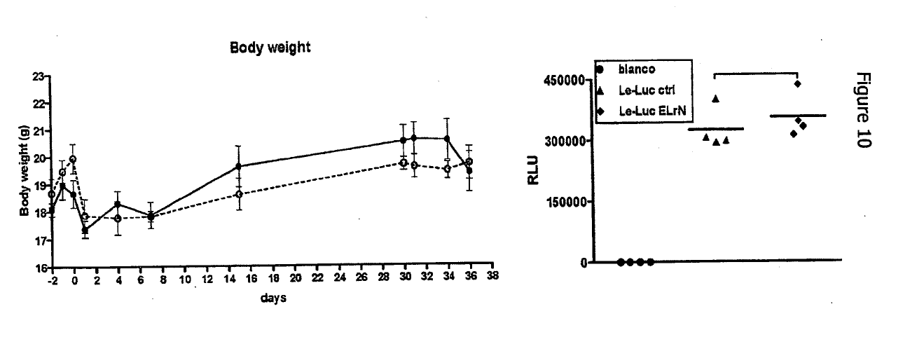

FIG. 10 shows luminescent intensity of pre-implantation graft and animal body weight during in vivo experiments. (A) The body weight of animals transplanted with a control graft (dotted line) did not differ significantly from animals transplanted with a treated graft (full line) at any time point (n=4; p=0.12). (B) No significant difference was observed between control grafts and grafts pre-treated with EGF, LIF and Notch1-EC (indicated as ELrN) prior to implantation under the kidney capsule, when comparing luminescent signal intensity (expressed as Relative Luminescent Units) (n=4; p>0.05). Cells untransduced with the luciferase overexpression construct displayed only minimal luminescence.

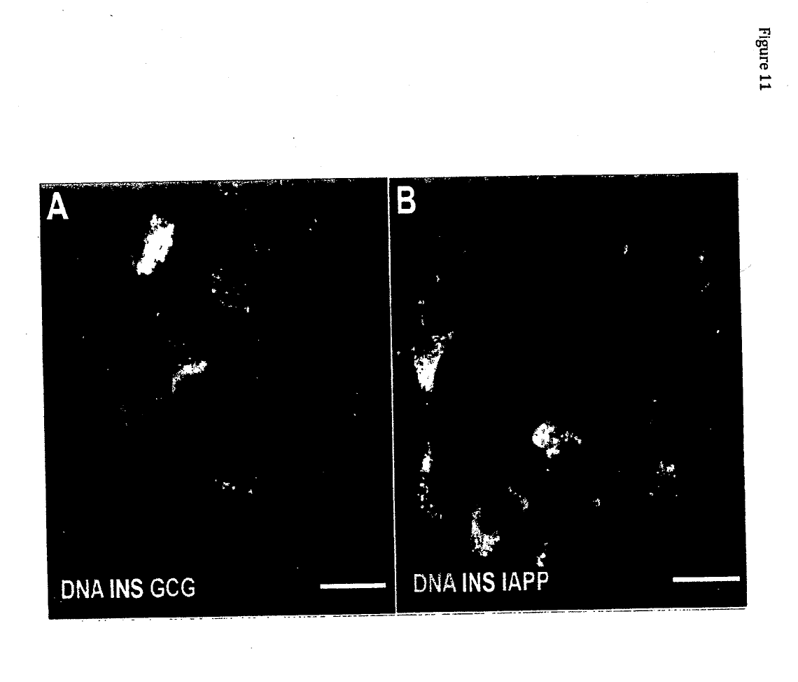

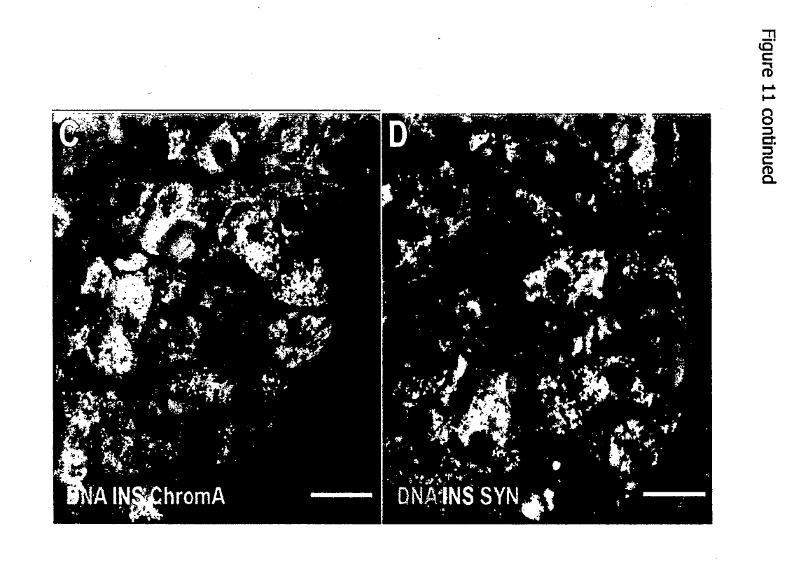

FIG. 11 is an illustration of phenotypical maturity of a graft. (A) Double staining using anti-insulin (green) and anti-glucagon (red) revealed the presence of very few alpha cells within the transplanted graft. Scale bar=10 μm. (B-D) Upon removal of the graft, the maturity of the beta cells was evaluated by immunohistochemistry. Double staining using anti-insulin (green) and anti-IAPP (red) (B), anti-chromograninA (red) (C) or anti-synaptophysin (red) (D) showed that almost all cells co-expressed both markers. In contrast to their in vitro phenotype, the acinar-derived beta cells underwent an in vivo maturation process post-implantation. Scale bar=10 μm.

### DETAILED DESCRIPTION OF THE INVENTION

Definitions

As used herein, the singular forms “a”, “an”, and “the” include both singular and plural referents unless the context clearly dictates otherwise. By way of example, “a cell” refers to one or more than one cells.

The terms “comprising”, “comprises” and “comprised of” as used herein are synonymous with “including”, “includes” or “containing”, “contains”, and are inclusive or open-ended and do not exclude additional, non-recited members, elements or method steps.

The term “about” as used herein when referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less from the specified value, insofar such variations are appropriate to perform in the disclosed invention.

The recitation of numerical ranges by endpoints includes all numbers and fractions subsumed within that range, as well as the recited endpoints.

All documents cited in the present specification are hereby incorporated by reference in their entirety. In particular, the teachings of all documents herein specifically referred to are incorporated by reference.

As used herein, the term “agent” broadly refers to any chemical (e.g., inorganic or organic, biochemical or biological substance, molecule or macromolecule (e.g., biological macromolecule), a combination or mixture thereof, a sample of undetermined composition, or an extract made from biological materials such as bacteria, plants, fungi, or animal cells or tissues. Preferred though non-limiting “agents” include nucleic acids, oligonucleotides, ribozymes, polypeptides or proteins, a peptides, peptidomimetics, antibodies and fragments and derivatives thereof, aptamers, chemical substances, preferably organic molecules, more preferably small organic molecules, lipids, carbohydrates, polysaccharides, etc., and any combinations thereof.

The terms “polypeptide” and “protein” are used interchangeably herein and generally refer to a polymer of amino acid residues linked by peptide bonds, and are not limited to a minimum length of the product. Thus, peptides, oligopeptides, polypeptides, dimers (hetero- and homo-), multimers (hetero- and homo-), and the like, are included within the definition. Both full-length proteins and fragments thereof are encompassed by the definition. The terms also include post-expression modifications of the polypeptide, for example, glycosylation, acetylation, phosphorylation, etc. Furthermore, for purposes of the present invention, the terms also refer to such when including modifications, such as deletions, additions and substitutions (e.g., conservative in nature), to the sequence of a native protein or polypeptide.

The term “peptide” as used herein preferably refers to a polypeptide as used herein consisting essentially of ≦50 amino acids, e.g., ≦45 amino acids, preferably ≦40 amino acids, e.g., ≦35 amino acids, more preferably ≦30 consecutive amino acids, e.g., ≦25, ≦20, ≦15, ≦10 or ≦5 amino acids.

The term “nucleic acid” as used herein means a polymer of any length composed essentially of nucleotides, e.g., deoxyribonucleotides and/or ribonucleotides. Nucleic acids can comprise purine and/or pyrimidine bases, and/or other natural, chemically or biochemically modified (e.g., methylated), non-natural, or derivatised nucleotide bases. The backbone of nucleic acids can comprise sugars and phosphate groups, as can typically be found in RNA or DNA, and/or one or more modified or substituted (such as, 2′-O-alkylated, e.g., 2′-O-methylated or 2′-O-ethylated; or 2′-O,4′-C-alkynelated, e.g., 2′-O,4′-C-ethylated) sugars or one or more modified or substituted phosphate groups. For example, backbone analogues in nucleic acids may include phosphodiester, phosphorothioate, phosphorodithioate, methylphosphonate, phosphoramidate, alkyl phosphotriester, sulfamate, 3′-thioacetal, methylene (methylimino), 3′-N-carbamate, morpholino carbamate, and peptide nucleic acids (PNAs).

The term “nucleic acid” further specifically encompasses DNA, RNA and DNA/RNA hybrid molecules, specifically including hnRNA, pre-mRNA, mRNA, cDNA, genomic DNA, gene, amplification products, oligonucleotides, and synthetic (e.g. chemically synthesised) DNA, RNA or DNA/RNA hybrids. The terms “ribonucleic acid” and “RNA” as used herein mean a polymer of any length composed of ribonucleotides. The terms “deoxyribonucleic acid” and “DNA” as used herein mean a polymer of any length composed of deoxyribonucleotides. The term “DNA/RNA hybrid” as used herein mean a polymer of any length composed of one or more deoxyribonucleotides and one or more ribonucleotides.

A nucleic acid can be naturally occurring, e.g., present in or isolated from nature, can be recombinant, i.e., produced by recombinant DNA technology, and/or can be, partly or entirely, chemically or biochemically synthesised. A nucleic acid can be double-stranded, partly double stranded, or single-stranded. Where single-stranded, the nucleic acid can be the sense strand or the antisense strand. In addition, nucleic acid can be circular or linear.

The term “oligonucleotide” as used herein denotes single stranded nucleic acids (nucleotide multimers) of greater than 2 nucleotides in length and preferably up to 200 nucleotides in length, more preferably from about 10 to about 100 nucleotides in length, even more preferably from about 12 to about 50 nucleotides in length. Oligonucleotides can be synthesised by any method known in the art, e.g., by chemical or biochemical synthesis, e.g., solid phase phosphoramidite chemical synthesis, or by in vitro or in vivo expression from recombinant nucleic acid molecules, e.g., bacterial or retroviral vectors.

Method of Culturing a Population of Cells Comprising Dedifferentiated Exocrine Pancreatic Cells

In a first aspect, the present invention is directed to a method for the generation of insulin-producing beta cells from a population of mammalian cells comprising dedifferentiated exocrine pancreatic cells. In such method use is made of agents that can inhibit Notch1 signaling pathway in the dedifferentiated exocrine pancreatic cells during the generation process.

More in particular, the invention provides an in vitro method of generating insulin-producing beta cells from a population of mammalian cells comprising dedifferentiated exocrine pancreatic cells comprising the step of culturing said dedifferentiated exocrine pancreatic cells in a culture medium
in the presence of A) at least Notch1 signaling pathway inhibiting agent and B) at least one ligand of the gp130 receptor, or
in the presence of A) at least Notch1 signaling pathway inhibiting agent and B) at least one ligand of the EGF receptor, or
in the presence of A) at least Notch1 signaling pathway inhibiting agent and B) at least one ligand of the gp130 receptor and at least one ligand of the EGF receptor.

The terms “agent that is able to inhibit the Notch1 signaling pathway” and “Notch1 signaling pathway inhibiting agent” are used herein as synonyms and are intended to refer to agents that interfere with Notch1 signaling pathway either by physiological or an RNAi-based mode of action.

In a preferred embodiment, the invention provides an in vitro method for generating insulin-producing beta cells from dedifferentiated exocrine pancreatic cells comprising the step of culturing said dedifferentiated exocrine pancreatic cells in a culture medium in the presence of
at least one agent that is able to inhibit the Notch1 signaling pathway in said dedifferentiated exocrine pancreatic cells, and
at least one ligand of the gp130 receptor and/or at least one ligand of the EGF receptor.

More in particular, the present invention provides a method comprising the steps of:
a) providing a population of dedifferentiated exocrine pancreatic cells in a culture medium,
b) adding at least one agent that is able to inhibit the Notch1 signaling pathway in said dedifferentiated exocrine pancreatic cells to said culture medium,
c) adding at least one ligand of the gp130 receptor and/or at least one ligand of the EGF receptor to said culture medium, and
d) incubating said dedifferentiated exocrine pancreatic cells in said culture medium.

The invention provides a method wherein more than 20%, preferably more than 30% and more preferably more than 40% of dedifferentiated exocrine pancreatic cells are insulin-producing cells having markers of mature beta cells selected from the group comprising C-peptide-I, Glut-2, Pdx-1, insulin and synaptophysin. In an embodiment the invention provides an in vitro method for generating insulin-producing beta cells from dedifferentiated exocrine acinar cells which comprises the step of culturing said dedifferentiated exocrine acinar cells in a culture medium in the presence of at least one agent that is able to inhibit the Notch1 signaling pathway in said dedifferentiated exocrine pancreatic cells, and at least one ligand of the gp130 receptor and at least one ligand of the EGF receptor. More in particular, in such embodiment a present method according to the invention comprises the steps of:
a) providing a population of dedifferentiated exocrine acinar cells in a culture medium,
b) adding at least one agent that is able to inhibit the Notch1 signaling pathway in said dedifferentiated exocrine pancreatic cells to said culture medium,
c) adding at least one ligand of the gp130 receptor and at least one ligand of the EGF receptor to said culture medium, and
d) incubating said dedifferentiated exocrine acinar cells in said culture medium.

The present method is directed to the neogenesis of insulin-producing cells, i.e. it relates to the generation of new insulin-producing cells from insulin-negative precursor cells in the exocrine part of the pancreas. Such method is clearly different from methods for purifying insulin-producing cells that are present in the endocrine part of pancreas tissue, such as disclosed for instance in WO 03/093458. The method referred to in WO 03/093458 is merely is a method to isolate pre-existing beta cells from the pancreas. In contrast, the present invention provides a method to generate/produce new beta cells starting from a cell preparation that is devoid of beta cells as it consists of pancreatic cells derived from the exocrine part of the pancreas which per definition does not contain insulin-producing cells. Much more beta cells can be obtained by generating new beta cells from exocrine pancreatic cells, then by isolating endogenous beta cells from the pancreas.

Pancreatic Cells

The islets or islet of Langerhans are special groups of cells in the pancreas. They make and secrete hormones that help the body break down and use food. These cells sit in clusters in the pancreas. There are five types of cells in an islet: beta cells, alpha cells, delta cells, which make somatostaton, and PP cells and D1 cells.

“Beta cells” are generally known as specialized cells found in clusters (islets) in the pancreas. Beta cells regulate glucose levels in the bloodstream by making insulin, monitoring glucose levels, and secreting insulin in response to elevated glucose levels. Together with glucagon secreting alpha cells, they form the majority of the endocrine cell population of the pancreas.

The term “dedifferentiated exocrine pancreatic cells” refers to those cells which are to a lesser or greater extent dedifferentiated and have re-acquired embryonic plasticity. Typical features of differentiated cells which have been lost by the dedifferentiated cells are the expression of amylase and other zymogens, such as pancreatic trypsinogen, trypsin and lipase, the insulin-transactivating transcription factor Pdx-1, the beta-cell specific glucose transporter Glut-2, and the C-peptide-I component of unprocessed proinsulin. Typical features of the embryonic plasticity which have been acquired by the dedifferentiated cells are the expression of cytokeratins 7,19 and/or 20. In addition there can be expression of the cholecystokinin B CCKB-receptors for gastrin, the neuroendocrine markers PGP9.5 (neuron-specific ubiquitin c-terminal hydrolase) and the Notch-1 receptor. Typical pancreatic cell types which can be dedifferentiated according to the present invention are acinar cells, duct cells and non-endocrine islet cells.

The term “redifferentiated beta cells” as used herein refers to beta cells which can be obtained by culturing dedifferentiated exocrine pancreatic cells under specific conditions as defined herein, and which have to a lesser or greater extent adopted the beta cell phenotype, including the capacity to secrete insulin. Typical dedifferentiated cells pancreatic cell types which can be redifferentiated according to the present invention are dedifferentiated acinar cells, duct cells and non-endocrine islet cells. It shall be noted that the expressions “redifferentiated beta cells” or “cells adopting a beta cell phenotype” or “insulin-producing cells” or “insulin secreting cells” or “insulin-positive cells” are used herein as synonyms and refer to beta cells that are generated from dedifferentiated exocrine pancreatic cells.

Preferably the dedifferentiated exocrine pancreatic cells which are used to generate insulin-producing beta cells are dedifferentiated exocrine duct cells, acinar cells or islet cells, and most preferably dedifferentiated exocrine acinar cells. Also mixtures of these types of cells, or also cell populations, comprising a certain ratio of one or more of the cells types consisting of the group consisting of duct cells, acinar cells and islet cells can be used in the present invention.

Pancreatic cells, such as those enumerated above, which can be used according to the methods of the present invention are all types of mammalian cells including rodent, porcine, monkey and human cells.

The present invention makes use of the exocrine fraction which is normally discarded after isolation of islets of Langerhans from the pancreas of humans and other mammals. In certain embodiments pancreatic exocrine cells are derived from adult, postnatal or prenatal pancreas. In a certain embodiment, the redifferentiated endocrine pancreatic cells are used for transplantation into a different species. In another embodiment redifferentiated endocrine cells are used for transplantation into a different individual of the same species. In yet another embodiment cells, exocrine pancreatic cells are obtained from an individual and the redifferentiated endocrine cells are used for transplantation into the same individual. Dedifferentiated cells can be maintained in culture for longer periods up to 14 days. Alternatively, the dedifferentiated cells are frozen and stored.

The Notch Signaling Pathway

The Notch signaling pathway is a highly conserved cell signaling system present in most multicellular organisms. The Notch signaling pathway is important for cell-cell communication, which involves gene regulation mechanisms that control multiple cell differentiation processes during embryonic and adult life.

Four different Notch receptors, referred to as Notch1 to Notch4, have been identified in vertebrates. The Notch1 receptor is a transmembrane receptor protein. It is a hetero-oligomer composed of a large extracellular portion which associates in a calcium dependent, non-covalent interaction with a smaller piece of the Notch protein composed of a short extracellular region, a single transmembrane-pass, and a small intracellular region.

Ligand proteins, such as Jagged1, Jagged2, Dll1, Dll4, binding to the extracellular domain of the Notch1 receptor induce proteolytic cleavage and release of the intracellular domain, which enters the cell nucleus to alter gene expression. More in particular, ligand-binding results in proteolytic cleavage of Notch receptors to release the signal-transducing Notch intracellular domain (NICD). NICD migrates into the nucleus and associates with the nuclear proteins of the RBP-Jkappa family (also known as CSL or CBF1/Su(H)/Lag-1). RBP-Jkappa, when complexed with NICD, acts as a transcriptional activator, and the RBP-Jkappa-NICD complex activates expression of primary target genes of Notch signaling such as the Hairy/Enhancer-of-split (HES) family of transcriptional repressors.

The above terms “Notch1” or “Notch1 receptor” are used herein to refer to polypeptides from any organism where found, and particularly from animals, preferably vertebrates, more preferably mammals, including humans and non-human mammals such as rodents (rat, mouse). The terms “Notch1” or “Notch1 receptor as used herein refer to said enzymes when forming part of a living organism, organ, tissue, and/or cell, as well as when at least partly isolated therefrom, reconstituted, etc.

The term “HES genes” as used herein are intended to refer to genes encoding proteins of the Hairy/Enhancer-of-split (HES) family, such as but not limited to Hes1, Hes5, Hes7, Hey1 Hey2. Several HES genes have been described in vertebrates, which function in different tissues as nuclear effectors of Notch signaling. Genes of the Hairy/Enhancer-of-split (HES) family encode basic-Helix-Loop-Helix proteins that function as nuclear effectors of Notch signaling to regulate the transcriptional activity of several Notch (secondary) target genes.

By “encoded” or “encoding” is meant that a nucleic acid sequence or its part corresponds, by virtue of the genetic code of an organism in question, preferably mammalian, e.g., human, to a particular amino acid sequence, e.g., the amino acid sequence of a particular polypeptide or protein. By means of example, a nucleic acid sequence “encoding” a particular polypeptide or protein may include naturally-occurring genomic, hnRNA, pre-mRNA, mRNA or therefrom obtained cDNA for the said polypeptide or protein, or may include recombinant counterparts or variants of such naturally-occurring nucleic acid sequences.

Liqands of the gp130 and/or of the EGF Receptor

The present method involves the use of one or more ligands of the gp130 receptor (glycoprotein 130 receptor). The gp130 receptor as used herein refers to a molecule comprising one IG-like domain, five FNIII (extracellular) domains, a transmembrane domain and six intracullular tyrosine residues. “Ligands of the gp130 receptor” are intended to refer to molecules capable of interacting with one or more of these domains of the gp130 receptor.

Naturally or modified proteins which are a ligand for the gp130 receptor and activate the downstream pathway that can be used for the methods of the present invention include but are not limited tot IL-6 (interleukin-6), IL-11 (interleukin-11), OSM (oncostatin M), CNTF (Ciliary Neurotrophic Factor), G-CSF (Granulocyte-colony stimulating factor), CT-1 (cardiotrophin-1), IL-12 (interleukin-12), Leptin, and LIF (Leukemia inhibitory factor). In a preferred embodiment the ligand of the gp130 receptor is LIF which binds to the gp130 receptor and activates a downstream pathway. LIF is a pleiotropic cytokine for which a function in pancreatic development has so far not been described. It is a well-known regulator of stem cell proliferation and differentiation and is widely used to prevent differentiation of embryonic stem cells. Truncated or mutated versions of LIF which retain the activity of binding and activating the gp130 receptor can be used as an alternative for the methods of the present invention.

The present method also involves the use of one or more ligands of the EGF receptor (epidermal growth factor receptor). A “ligand of the EGF receptor” as used herein refers to a molecule, e.g. a protein, having a conserved structure with six cystein residues which may form three intra-molecular disulphide bounds.

Naturally or modified proteins which are a ligand for EGF receptor that can be used for the methods of the present invention include but are not limited to Transforming Growth Factor-alpha, amphiregulin, betacellulin and PoxVirus Growth Factor and EGF (epidermal growth factor). In a preferred embodiment the ligand of the EGF receptor is EGF which binds to the EGF receptor (EGFR) and activates a downstream pathway. Consequently truncated or mutated versions of EGF which retain the activity of binding and activating the EGF receptor can be used as an alternative for the methods of the present invention.

Agents Inhibiting the Notch1 Signalling Pathway

The present cultivation methods are at least in part based on the use of an agent that is able to inhibit the Notch1 signaling pathway in said dedifferentiated exocrine pancreatic cells during cultivation thereof.

The term “can”, as in, e.g., “can inhibit Notch1 signaling pathway” or “can inhibit Notch1 activity”, is synonymous to “is capable of” and signifies that an entity, e.g., an agent, has the ability to achieve the recited effect or action, e.g., when added to a culture medium wherein a population of cells comprising dedifferentiated exocrine pancreatic cells is cultured.

In accordance with the present invention, “inhibition of the Notch1 signaling pathway” includes physical interference as well as RNAi based interference with one or more aspects of the Notch1 signaling pathway by means of one or more agents as explained below.

The term “inhibit” encompasses any extents of inhibition of one or more aspects of Notch1 signaling pathway, e.g. activity of the Notch1 receptor and/or of downstream effectors thereof such as the activity of HES gene products (e.g. Hes1). For example, inhibition may be by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, when the Notch1 receptor is exposed to an agent as defined herein.

### Notch1 Inhibiting Agent

In a first embodiment the agents inhibiting the Notch1 signalling pathway used in accordance with the present invention are agents that physically interfere with Notch1 signaling. An “agent capable of physically interfering with Notch1 signaling” is herein also denoted as “Notch1 inhibiting agent” and can be a chemical substance, preferably an organic molecule, preferably a peptide or a polypeptide.

Notch1 inhibiting agents used in accordance with the invention may bind to the Notch1 receptor. The term “binding” as used herein generally refers to a physical association, preferably herein a non-covalent physical association, between molecular entities, e.g., between a “ligand” (generally referring to any agent, e.g., a substance or molecule) and a “receptor” (generally referring to any molecule). Preferably, a “receptor” may be a polypeptide or protein, such as, e.g., Notch1 or variants or fragments thereof. Preferably, a “ligand” may be, e.g., a polypeptide or protein, an antibody, a peptide, a peptidomimetic, an aptamer, a chemical substance (preferably an organic molecule, more preferably a small organic molecule), a lipid, a carbohydrate, a nucleic acid, etc.

Alternatively or in combination therewith such Notch1 inhibiting agent may obstruct or reduce binding of the Notch1 receptor with one or more of its ligands, such as e.g. Jagged1 and Dll4, and thus reduce interaction of the Notch1 receptor and its ligand(s), and therefore also reduce Notch1 receptor activity.

In a preferred embodiment, a “Notch1-inhibiting agent” as defined herein is intended to refer to an allosteric inhibitor or a chemical inhibitor capable of inhibiting the Notch1 receptor.

An allosteric inhibitor of the Notch 1 receptor refers to a molecule, e.g. a protein, able to inhibit the Notch1 receptor by binding to the Notch1 receptor and thus preventing binding of the Notch1 receptor with one of its ligand e.g. Jagged1, Jagged 2, Dll1, Dll4. More preferably such allosteric inhibitor is a molecule, e.g. a protein, having extracellular domains containing 36 EGF modules in tandem. An example of a suitable allosteric Notch1-inhibiting agents includes but is not limited to Notch1-EC.

A chemical inhibitor refers to a molecule able to inhibit one of the cleaving enzymes of Notch 1, e.g. TACE or gamma-secrase cleaving.

According to a preferred embodiment the Notch1-inhibiting agent is an allosteric inhibitor of the Notch1 receptor and preferably is Notch1-EC, such as human or humanised Notch1-EC.

According to another embodiment the Notch1-inhibiting agent is a chemical inhibitor of the Notch1 receptor, such as but not limited to DAPT, Compound E, and L-685,458.

RNA Interference Agent

In another embodiment agents inhibiting the Notch1 signalling pathway used in accordance with the present invention are agents capable of reducing the level of expression” of Notch 1 and/or Hes genes. In preferred embodiments, agents capable of reducing the expression of Notch 1 and/or Hes genes can be chosen from the group comprising a chemical substance, preferably an organic molecule, more preferably an agent capable of causing RNA interference, also denoted as “RNA interference agent” or “RNAi molecule” herein. An agent capable of reducing the expression of Notch 1 and/or Hes genes, is capable of causing RNA interference with the respective transcripts, preferably mRNAs.

When an agent, e.g., a substance or molecule, is said to “reduce the expression” of Notch 1 and/or Hes genes, this generally means that administration of the said substance to a cell, tissue or an organism, causes Notch 1 and/or Hes genes to be expressed at a level relatively lower than if the said substance had not been administered. Such reduction of expression can be observed and quantified, e.g., at the level of heterogeneous nuclear RNA (hnRNA), precursor mRNA (pre-mRNA), mRNA, cDNA and/or the protein of HADHSC. Suitable methods to detect and quantify expression are known in the art and include, without limitation, Northern blotting, quantitative RT-PCR, Western blotting, ELISA, RIA, immunoprecipitation, etc. The term encompasses any extent of reduction of expression, such as, by way of example, reduction of expression by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, e.g., as measured in gross mass and/or at the level of individual cells.

“RNA interference” or “RNAi” is a term initially applied to a phenomenon observed in plants and worms where double-stranded RNA (dsRNA) blocks gene expression in a specific and post-transcriptional manner. Consequently, RNAi refers generally to the process of sequence-specific post-transcriptional gene silencing in animals mediated by short interfering nucleic acids (siNA), preferably by short interfering RNAs (siRNAs). RNAi provides a useful method of inhibiting gene expression in vitro or in vivo.

RNA interference agents (RNAi molecules) may include any of short interfering nucleic acid (siNA), short interfering RNA (siRNA), double-stranded RNA (dsRNA), micro-RNA (miRNA), and short hairpin RNA (shRNA) molecules capable of mediating RNA interference (RNAi) against the expression of Notch 1 and/or Hes genes.

In the present context, the expression “dsRNA” relates to double stranded RNA capable of causing RNA interference. In accordance with the present invention, any suitable double-stranded RNA fragment capable of directing RNAi or RNA-mediated gene silencing of a target gene can be used. As used herein, a “double-stranded ribonucleic acid molecule (dsRNA)” refers to any RNA molecule, fragment or segment containing two strands forming an RNA duplex, notwithstanding the presence of single stranded overhangs of unpaired nucleotides. The double-stranded RNA comprises annealed complementary strands, one of which has a nucleotide sequence which corresponds to a target nucleotide sequence (i.e. to at least a portion of the mRNA transcript) of the target gene to be down-regulated. The other strand of the double-stranded RNA is complementary to this target nucleotide sequence.

The double-stranded RNA need only be sufficiently similar to the mRNA sequence of the target gene to be down-regulated that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and a nucleotide sequence of the dsRNA sequence is no more than 1 in 5 basepairs, or 1 in 10 basepairs, or 1 in 20 basepairs, or 1 in 50 basepairs.

According to the invention, the “dsRNA” or “double stranded RNA”, whenever said expression relates to RNA that is capable of causing interference, may be formed form two separate (sense and antisense) RNA strands that are annealed together. Alternatively, the dsRNA may have a foldback stem-loop or hairpin structure wherein the two annealed strands of the dsRNA are covalently linked. In this embodiment, the sense and antisense strands of the dsRNA are formed from different regions of a single RNA sequence that is partially self-complementary.

As used herein, the term “RNA interfering agent” or “RNAi molecule” is a generic term referring to double stranded RNA molecules including small interfering RNAs (siRNAs), hairpin RNAs (shRNAs), and other RNA molecules which can be cleaved in vivo to form siRNAs. RNAi molecules can comprise either long stretches of dsRNA identical or substantially identical to the target nucleic acid sequence or short stretches of dsRNA identical or substantially identical to only a region of the target nucleic acid sequence.

The subject RNAi molecules can be “small interfering RNAs” or “siRNAs.” siRNA molecules are usually synthesized as double stranded molecules in which each strand is around 19-30 nucleotides in length, and even more preferably 21-23 nucleotides in length. The siRNAs are understood to recruit nuclease complexes and guide the complexes to the target mRNA by pairing to the specific sequences. As a result, the target mRNA is degraded by the nucleases in the protein complex. In a particular embodiment, the siRNA molecules comprise a 3′ hydroxyl group. In certain embodiments, the siRNA molecules can be generated by processing of longer double-stranded RNAs, for example, in the presence of the enzyme dicer.

Alternatively, the RNAi molecule is in the form of a hairpin structure, named as hairpin RNA or shRNA. The hairpin RNAs can be synthesized exogenously or can be formed by transcribing from RNA polymerase III promoters in vivo. Preferably, such hairpin RNAs are engineered in cells or in an animal to ensure continuous and stable suppression of a desired gene. It is known in the art that siRNAs can be produced by processing a hairpin RNA in the cell.

In a preferred embodiment, the agent capable of causing RNA interference with Notch1 or a Hes gene, preferably Hes1 used in the present method is a RNA interfering agent chosen from the group comprising short interfering nucleic acid (siNA), short interfering RNA (siRNA), double-stranded RNA (dsRNA), micro-RNA (miRNA), and short hairpin RNA (shRNA).

Preferably the present RNAi molecules are shRNA molecules directed against Notch1 and/or Hes genes, preferably Hes1.

In one specific embodiment, said RNA interfering agent is a shRNA having at least 50% sequence identity, preferably at least 70% sequence identity, more preferred at least 80% sequence identity, even more preferred at least 90, 92, 95, 96, 97, 98, 99, 100% sequence identity with Notchl mRNA. Preferably said RNA interfering agent is a shRNA having a sequence as represented in SEQ ID NO:1. In another specific embodiment, said RNA interfering agent is a shRNA having at least 50% sequence identity, preferably at least 70% sequence identity, more preferred at least 80% sequence identity, even more preferred at least 90, 92, 95, 96, 97, 98, 99, 100% sequence % sequence identity with the mRNA of a Hes gene, and preferably with Hes1 mRNA. Preferably said RNA interfering agent is a shRNA having a sequence as represented in SEQ ID NO:2. Sequence identity between two nucleotide sequences can be calculated by aligning the said sequences and determining the number of positions in the alignment at which the two sequences contain the same nucleic acid base vs. the total number of positions in the alignment.

Production of any above nucleic acid reagents, including RNAi molecules, can be carried out by chemical synthetic methods or by recombinant nucleic acid techniques, e.g., expressed from a vector in a cell, e.g., a viral vector, a eukaryotic expression vector, a gene therapy expression vector (i.e., in vivo), etc., or enzymatically synthesized, e.g., by in vitro transcription from a DNA template using a T7 or SP6 RNA polymerase.

Any above nucleic acid reagents, including RNAi molecules, can be purified using a number of techniques known to those of skill in the art. For example, gel electrophoresis can be used to purify nucleic acid reagents. Alternatively, non-denaturing methods, such as non-denaturing column chromatography, can be used to purify the molecules. In addition, chromatography (e.g., size exclusion chromatography), glycerol gradient centrifugation, affinity purification with antibody can be used to purify the molecules.

Depending on the precise nature of the agents capable of reducing the level of expression of Notch1 and/or Hes genes, these may be delivered to cells in vitro according to protocols commonly employed in the art. By means of example, there are several well-known methods of introducing (ribo)nucleic acids, e.g., RNAi, recombinant nucleic acids encoding an agent, e.g., an shRNA, into animal cells, any of which may be used in the present invention and which depend on the host.

The nucleic acid can be directly injected into the target cell/target tissue. Other methods include fusion of the recipient cell with bacterial protoplasts containing the nucleic acid, the use of compositions like calcium chloride, rubidium chloride, lithium chloride, calcium phosphate, DEAE dextran, cationic lipids or liposomes or methods like receptor-mediated endocytosis, biolistic particle bombardment (“gene gun” method), infection with viral vectors, electroporation, and the like. It shall be clear that also a combination of different above-mentioned delivery modes or methods may be used. In a preferred embodiment infection with viral vectors is preferred.

Concentrations

The terms “added”, “adding” or “addition” in the present invention refers to compounds, such as for instance EGF, LIF, or Notch1 inhibiting agents such as Notch1-EC, which are supplemented separately to the culture medium. It does not refer to unknown levels of compounds which are present in the medium due to secretion by the cells. It also does not refer to low amounts of compounds which are present in serum which is added to a basal growth medium.

Ligands of the gp130 or of the EGF receptor, such as for instance LIF or EGF, respectively, or Notch1 inhibiting agents such as Notch1-ECEGF used in the methods of the present invention for the redifferentiation can be from the same species as the species from which the pancreatic cells are isolated but can also be from another species. Said ligands or agents are preferably obtained from mammals such as but not limited to humans, primate and non primate monkeys, rodents such as hamster, mouse and rat, rabbits, sheeps, cows and other cattle, dogs and porks. When using such ligands or agents from another species as the species from which the pancreatic cells are isolated the sequence of such ligands or agents from one species can be modified in order to acquire the desired binding and activation properties on the cell population obtained from another species. For in vivo purposes a ligand from a non-human mammal can be “humanised” in order to acquire activity within a human and to avoid an immune response by the human immune system. Therefore, human or “humanised” ligands of the gp130, e.g. human or “humanised” LIF, human or “humanised” ligands of the EGF receptor, e.g. human or “humanised” EGF, respectively, or human or “humanised” Notch1 inhibiting agents, e.g. human or “humanized” Notch1-EC can ben used in the present methods.

Concentrations of added ligands of the gp130 or ligands of the EGF receptor or of Notch1 inhibiting agents in the culture medium are in the ng/ml range. Generally, the one or more ligands of the gp130 receptor, the one or more of the ligands of the EGF receptor, and the one or more Notch1 inhibiting agents are added to the culture medium in a concentration between 1 and 10 000 ng/ml. More particularly, concentrations of added ligands and Notch1 inhibiting agents in the culture medium may vary from about 1, 10, 25, 50, 100, 250, 500, up to 1000 ng/ml. In a specific embodiment the concentration of added compounds for each compound separately varies between 10 and 100 ng/ml, and preferably between 20 and 100 ng/ml.

In a specific embodiment one or more ligands of the gp130 receptor, and preferably LIF, are added to the culture medium in a concentration between 1 and 10 000 ng/ml, preferably between 10 and 100 ng/ml or between 10 and 25 ng/ml or between 100 and 500 ng/ml.

In another specific embodiment one or more ligands of the EGF receptor, and preferably EGF, are added to the culture medium in a concentration between 1 and 10 000 ng/ml, preferably between 10 and 100 ng/ml or between 10 and 25 ng/ml or between 100 and 500 ng/ml.

In yet another specific embodiment one or more Notch1 inhibiting agents, and preferably Notch1-EC, are added to the culture medium in a concentration between 1 and 70 μg/ml, preferably between 3 and 60 μg/ml or between 5 and 50 μg/ml.

Further Culture Conditions

Preferably, in accordance with the present method the dedifferentiated exocrine pancreatic cells are cultured as monolayer cell cultures.

The dedifferentiated pancreatic cells used in this method can be depleted from beta cells prior to the incubation into this medium. In a preferred embodiment, the method therefore further comprises the step of depleting the population of cells comprising dedifferentiated exocrine pancreatic cells of beta cells prior to culturing thereof, e.g. by treating said cells with alloxan.

According to a particular embodiment, the method further comprises the step of adding bFGF (basic fibroblast growth factors) to said culture medium.

According to a particular embodiment the medium is free from KGF (keratinocyte growth factor) or a gastrin/CCK receptor ligand.

In particular embodiments the incubation step in the present method is performed during 7,6, 5 or even less than 5 days namely 4 or 3 days. Incubation is done at temperature of between 35 and 38° C., preferably at 37° C.

According to yet another embodiment, in order to reduce the growth of fibroblast cells, dedifferentiation and redifferentiation is performed in the presence of gentamycine.

Cell Population

The invention further provides a population of mammalian pancreatic cells comprising insulin-positive cells. It shall be noted that the expressions “insulin-producing cells” or “insulin-secreting cells” or “insulin-positive cells” are used herein as synonyms and refer to beta cells that are generated from dedifferentiated exocrine pancreatic cells and that are phenotypically characterized by their secretory capacity, in particular their ability to produce and secrete insulin. More in particular the above referred cells are characterised in that they produce and secrete insulin, c-peptide, Glut-2 (Glucose transporter-2), Pdx1 (Pancreatic and duodenal homeobox 1), and synaptophysin. “Insulin-positive cells” according to the invention are further functionally characterised by having glucose sensing capacity.

The invention thus provides a population of mammalian pancreatic cells comprising insulin-producing beta cells, wherein said insulin-producing beta cells are generated from dedifferentiated exocrine pancreatic cells, for instance acinar cells, duct cells and non-endocrine islet cells, and preferably from exocrine acinar cells. The invention thus provides a population of mammalian pancreatic cells comprising dedifferentiated exocrine pancreatic cells, for instance acinar cells, that have adopted a beta cell phenotype and that produce insulin. In one embodiment, this population of mammalian pancreatic cells comprises more than 20%, preferably more than 25%, preferably more than 30%, preferably more than 35%, preferably more than 40%, preferably more than 50% of insulin-positive cells. The invention provides a population of mammalian pancreatic cells comprising more than 20%, preferably more than 30% and more preferably more than 40% of dedifferentiated exocrine pancreatic cells that are generated from dedifferentiated exocrine pancreatic cells, for instance acinar cells, and that are insulin-producing cells having markers of mature beta cells such as those disclosed herein. In another embodiment, this population of mammalian pancreatic cells, after exposure to 20 mM glucose for 2 hours at 37° C. in HamF10 medium secretes at least half of the amount of insulin that is secreted by normal, endogenous, beta cells under identical conditions. In another embodiment, the population of mammalian pancreatic cells, after exposure to 20 mM glucose for 2 hours at 37° C. in HamF10 medium shows at least a 2 fold increase in insulin secretion when compared to the insulin secretion prior to said exposure to glucose. In another embodiment, the population of mammalian pancreatic cells after exposure to 20 mM glucose for 2 hours at 37° C. in HamF10 medium secretes insulin at a concentration of at least 2 pg insulin/cell. Cells are obtained having a secretion rate of at least 1 pg insulin/cell/h.

Another aspect of the present invention relates to a population of mammalian pancreatic cells comprising mammalian insulin-producing cells are characterised in that they have markers of mature (normal) beta cells, and for instance markers selected from the group comprising C-peptide-I, Glut-2, Pdx-1, insulin, and synaptophysin.

Redifferentiated beta cells can be distinguished from redifferentiated duct-like cells since redifferentiated duct-like cells do not produce insulin and do not comprise markers of mature beta cells such as C-peptide, Glut-2, synaptophysin or Pdx1.

Redifferentiated beta cells according to the invention may be temporarily binuclear. This permits to distinguish these cells by using FACS (fluorescence-activated cell sorting).

The present invention also relates to a population of mammalian pancreatic cells which is obtainable or obtained by any of the embodiments of the above-described redifferentation method.

Applications

A further object of the invention are pharmaceutical compositions which comprise a therapeutically effective amount of a population of mammalian pancreatic cells comprising insulin-producing pancreatic cells and preferably (redifferentiated) insulin-producing beta cells as defined herein which are obtainable by the method of the present invention and at least one pharmaceutically acceptable carrier, i.e., one or more pharmaceutically acceptable carrier substances and/or additives, e.g., buffers, carriers, excipients, stabilisers, etc.

In a further aspect, the invention therefore relates to a pharmaceutical composition comprising a therapeutically active amount of a population of mammalian pancreatic cells comprising (redifferentiated) insulin-producing beta cells as defined herein which are obtainable by the method of the present invention and at least one pharmaceutically acceptable carrier.

The term “therapeutically effective amount” as used herein means that population of cells comprising insulin-producing pancreatic cells that elicits the biological or medicinal response in a tissue, system, animal or human that is being sought by a researcher, veterinarian, medical doctor or other clinician.

The term “pharmaceutically acceptable” as used herein is consistent with the art and means compatible with the other ingredients of a pharmaceutical composition and not deleterious to the recipient thereof. Suitable pharmaceutically acceptable carriers are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

The present invention shows the in vitro redifferentiation of dedifferentiated exocrine cells in the presence of compounds such as the combination of LIF and EGF together with Notch1-EC or other Notch1 inhibiting agents as defined herein, or with RNA interfering agent(s) as defined herein.

In alternative embodiments the redifferentiation of cells is envisaged to happen within an individual to be treated. As an example, dedifferentiated cells are embedded within a biodegradable matrix further comprising compounds allowing a time and concentration controlled matrix comprising for example LIF and EGF together with Notch1-EC or other Notch1 inhibiting agents as defined herein, or together with RNA interfering agent(s) as defined herein, which allows the in vivo differentiation of dedifferentiated exocrine pancreatic cells. After degradation of the matrices, the differentiated cells are released. In a particular embodiment the cells are first treated with differentiating growth factors for a limited time in vitro and afterwards implanted in the presence of growth factors for further in vivo differentiation.

In an example, the invention may provide dedifferentiated cells as defined herein which are embedded within a biodegradable matrix and further comprises a) LIF and EGF together with Notch1-EC, or b) LIF and EGF and a shRNAi having a sequence as represented in SEQ ID NO:1 or c) LIF and EGF and a shRNAi having a sequence as represented in SEQ ID NO:2.

In a further aspect, the invention relates to a population of mammalian pancreatic cells comprising redifferentiated insulin-producing beta cells as defined herein or a pharmaceutical composition comprising such population as defined herein for use as a medicament. In a particular embodiment, the medicament is used for the treatment of diabetes type 1 or type 2.

The present invention also relates to a combination of A) a human or humanised ligand of a EGF receptor, and B) a human or humanised ligand of the gp130 receptor, and C) a human or humanised Notch1 inhibiting agent for use as a medicament. The present invention also relates to a combination of A) a human or humanised ligand of a EGF receptor, and B) a human or humanised ligand of the gp130 receptor, and C) a RNA interfering agent(s) as defined herein for use as a medicament. The present invention may also relate to a combination of A) a human or humanised ligand of a EGF receptor, and B) a human or humanised ligand of the gp130 receptor, and C) a human or humanised Notch1 inhibiting agent and D) one or more RNA interfering agents as defined herein for use as a medicament.

In yet another aspect, the invention relates to the use of a population of mammalian pancreatic cells comprising redifferentiated insulin-producing beta cells as defined herein or a pharmaceutical composition comprising such population as defined herein for the preparation of a medicament, in particular for the preparation of a medicament for the treatment of diabetes type 1 or type2.

The present invention relates to the use of a combination of A) a human or humanised ligand of a EGF receptor, and B) a human or humanised ligand of the gp130 receptor, and C) a human or humanised Notch1 inhibiting agent for the preparation of a medicament, in particular for the preparation of a medicament for the treatment of diabetes type 1 or type2. The present invention also relates to the use of a combination of A) a human or humanised ligand of a EGF receptor, and B) a human or humanised ligand of the gp130 receptor, and C) a RNA interfering agent(s) as defined herein for the preparation of a medicament, in particular for the preparation of a medicament for the treatment of diabetes type 1 or type2. The present invention also relates to a combination of A) a human or humanised ligand of a EGF receptor, and B) a human or humanised ligand of the gp130 receptor, and C) a human or humanised Notch1 inhibiting agent, and D) one or more RNA interfering agents as defined herein for the preparation of a medicament, in particular for the preparation of a medicament for the treatment of diabetes type 1 or type2.

The invention also relates to a population of mammalian pancreatic cells comprising redifferentiated insulin-producing beta cells as defined herein or a pharmaceutical composition comprising such population as defined herein for use for the treatment of diabetes type 1 or type2.

The present invention further relates to a combination of a human or humanised ligand of a EGF receptor, and a human or humanised ligand of the gp130 receptor, and a human or humanised Notch1 inhibiting agent for use for the treatment of diabetes type 1 or type2. The present invention further relates to a combination of a human or humanised ligand of a EGF receptor, and a human or humanised ligand of the gp130 receptor, and a RNA interfering agent(s) as defined herein for use for the treatment of diabetes type 1 or type2. The present invention also relates to a combination of A) a human or humanised ligand of a EGF receptor, and B) a human or humanised ligand of the gp130 receptor, and C) a human or humanised Notch1 inhibiting agent and D) one or more RNA interfering agents as defined herein for use for the treatment of diabetes type 1 or type2.

In one embodiment the human or humanised ligand of a EGF receptor is human EGF. In another embodiment the human or humanised ligand of the human gp130 receptor is human LIF. In yet another embodiment the human or humanised Notch1 inhibiting agent is Notch1-EC. In yet another embodiment the RNA interfering agent(s) is a shRNA having at least 50% sequence identity, preferably at least 70% sequence identity, more preferred at least 80% sequence identity, even more preferred at least 90% sequence identity with Notch1 mRNA, and preferably a shRNA having a sequence as represented in SEQ ID NO:1. In still another embodiment the RNA interfering agent(s) is a shRNA having at least 50% sequence identity, preferably at least 70% sequence identity, more preferred at least 80% sequence identity, even more preferred at least 90% sequence identity with the mRNA of a Hes gene, and preferably with Hes1 mRNA, and preferably is a shRNA having a sequence as represented in SEQ ID NO:2.

Further the invention relates to a method for the treatment of diabetes type 1 or type 2 comprising the step of administering an effective amount of a population of mammalian pancreatic cells comprising (redifferentiated) insulin-producing beta cells as defined herein or a pharmaceutical composition as defined herein to an individual in need of it.

The invention is further illustrated with examples that are not to be considered limiting.

### Examples

### Example 1

### Experimental Procedures

Animals

Male 10-12 week old Wistar rats (Charles River Laboratories) weighing 250-300 g were used for the isolation of cells from the pancreas. Pancreata were partially dissociated with collagenase and exocrine acini were purified by centrifugal elutriation as published before (Rooman et al., 2000 Diabetologia 43:907-914). All animal experimentation was approved by the Ethical Committee of the Free University of Brussels.

In Vivo Injections

Wheat Germ Agglutinin, coupled to Fluorescein-iso-thio-cyanate (FITC) (Invitrogen S.A.) was micro-injected directly into the pancreatic parenchyma at a dose of 150 mg/kg body weight. This dose was dissolved in 350 μl physiological fluid (Baxter S.A.) and injected at multiple loci in the pancreatic head and tail sections. The lectin was given 72 h before isolating the different cell types to allow binding to its target N-acetyl glucosamine and internalization in cytoplasmic storage compartments.

Cell Culture

Acinar cells were pre-cultured for 4 days in bacteriological Petri dishes (Nunc) in suspension culture in RPMI-1640 Glutamax-I medium supplemented with 10% fetal bovine serum (FBS, Invitrogen), penicillin (75 mg/l) (Continental Pharma) and antibiotics (Sigma, St Louis, Mo., USA). Geneticin Sulphate (50 μg/ml) (Sigma) was used to suppress fibroblast overgrowth in the culture. Medium was replaced daily during this pre-culture period. At the end of the pre-culture, cells were treated with alloxan (10 mM, Sigma) for 10 min at 37° C. to remove contaminating beta cells, and transferred to 24-well plates (Falcon, BD Biosciences) to form adherent cultures. Adherent monolayers were further cultured with RPMI supplemented with 1% FBS and antibiotics (controls). For growth factor stimulation of neogenesis this medium was supplemented with 50 ng/ml human recombinant epidermal growth factor (EGF) (Sigma,) and 40 ng/ml recombinant mouse leukemia inhibitory factor (LIF) (Sigma) (Baeyens et al. Diabetologia 48:49-57.). Stimulation of Notch signal transduction was performed using the recombinant human ligands Jagged1 (2 μg/ml, R&D Systems) and Dll4 (5 μg/ml, R&D Systems). For effective stimulation, the ligands were immobilized to the bottom of the multiwell plates prior to monolayer formation. Cell monolayers were analyzed after a culture period of 3 days in the latter media. At these concentrations, no toxic effects of the recombinant proteins were noted on the epithelial monolayers. Notch signaling inhibition was achieved using a recombinant form of the rat Notch1 extracellular domain (Notch1-EC, 10 μg/ml, R&D Systems). Efficient inhibition was found to be obtained using the soluble form of this recombinant protein. Proliferation was performed by bromo-deoxyuridin (BrdU) (Sigma) pulse labeling of 6 hours.

RT-PCR

Total RNA was isolated from cells and tissues using the GenElute Mammalian Total RNA miniprep kit (Sigma). For the semi-quantitative analysis of transcripts encoding rat NOTCH1, rat HES1, rat JAGGED1, rat DLL4, rat HES6 and rat ACTIN, the total RNA was reversed transcribed and amplified as described by the manufacturer (Invitrogen) with blanks in each assay. Primers were designed to anneal to the specific targets (Table 1).

Target genes selected from RT-PCR were further analyzed using qPCR to quantify their expression level. cDNA was prepared from 500 ng total RNA following DNase treatment and 10 ng RNA equivalent used for PCR with selected primers in the presence of SYBR Green (Invitrogen S.A.). A melt curve analysis was performed for each reaction. The expression levels were normalized to individual beta-actin (RNA input control) and to starting conditions (0 h treatment) (reference sample).

Primer Sequences

Table 1 represents a list of primer sequences.

| | | Sequence anti-sense |
|---|---|---|
| | Sequence sense primer | primer |
| Rat | CACACCAACGTGGTCTTCAAGC | TAGACAATGGAGCCACGGATGT |
| NOTCH1 | (SEQ ID NO: 4) | (SEQ ID NO: 5) |
| Rat | TTCAGCGAGTGCATGAACGA | TAGGTCATGGCGTTGATCTGG |
| HES1 | (SEQ ID NO: 6) | (SEQ ID NO: 7) |
| Rat | ACCACCTGCGAAGTGATTGAC | TGAATTTGCCTCCCGACTG |
| JAGGED1 | (SEQ ID NO: 8) | (SEQ ID NO: 9) |
| Rat | GGCAAACTGCAGAACCACACA | GACATGCCGGCTTTTCACTGT |
| DLL4 | (SEQ ID NO: 10) | (SEQ ID NO: 11) |
| Rat | GCTGCCATCCTTAAGTTTGC | GGTCATCAGGGTTACCATACAA |
| HES6 | (SEQ ID NO: 12) | (SEQ ID NO: 15) |
| Rat | ACTATCGGCAATGAGCGGTTC | AGAGCCACCAATCCACACAGA |
| ACTIN | (SEQ ID NO: 14) | (SEQ ID NO: 13) |

The cycling profile was: 1.5 min at 94° C. followed by 0.5 min at 94° C., 0.5 min at 60° C. and 1 min at 72° C. for 10 cycles and 0.5 min at 94° C., 0.5 min at 58° C. and 1 min at 72° C. for 16 to 20 cycles (total of 30 cycles for Notch1, 28 cycles for Hes1, 30 cycles for Jagged1, 30 cycles for D114, 30 cycles for Hes6 and 25 cycles for b-actin). Analysis of the amplified fragments was done on agarose gels stained with GelRed (VWR International). All analyses were performed at least three times.

Western Blotting

Total cellular protein fraction was extracted in Laemmli buffer (10% glycerol, 2.3% SDS, 0.125M Tris-HCl pH6,8). Concentrations were determined by the Quant-It protein assay method (Invitrogen S.A.) according to manufacturer's recommendations. Proteins were separated on SDS-polyacrylamide gels and electroblotted to low-fluorescence PVDF membranes. Western blotting was performed following the manufacturer's Qdot Werstern Blotting Kit protocol (Invitrogen S.A.). Loading of equal amount of proteins (25 μg per sample) was evaluated by detection of actin in the same blot. Comparison for the same protein between two different samples is always shown on the same gel. Visualisation of Qdot fluorescent signals was done by the Kodak GelLogic 100 system.

Antibodies were used as follows: polyclonal anti-Notch1-IC:1/30 (Cell Signaling Technologies); polyclonal anti-Hes1: 1/100 (T. Sudo) (Ito et al. 2000 Development 127:3913-3921); monoclonal anti-b-actin: 1/1000 (MP Biomedicals); donkey anti-rabbit-Qdot605 (Invitrogen): 1/1000; donkey anti-mouse-Qdot525: 1/1000 (Invitrogen).

Viral Constructs

Specific RNAi target sequences were developed using dedicated software available through the Whitehead Institute for Biomedical Research website (http://jura.wi.mit.edu/bio/) (Yuan et al. 2004 Nucleic Acids Res. 32:W130-W134). Corresponding oligo DNA molecules (Invitrogen) were cloned in the BglII and HindIII sites of the pSuper.basic vector (Oligoengine). Insert-containing clones were PCR-selected and sequenced. DNA from clones containing correct inserts was cut with EcoRI-ClaI, which released a DNA fragment containing the H1 promoter and the cloned oligo duplex, which was subsequently subcloned in the EcoRI-ClaI sites of the pTrip vector containing a DsRed reporter (Wiznerowicz and Trono 2003, J. Virol. 77:8957-8961). Screening for RNAi activity of the constructs was performed as follows: AR42J-B13 cells were plated at 70% confluency in 24 well plates at day 0. After allowing the cells to attach, 500 ng DNA of the appropriate pTrip constructs was transfected per well. After an additional 24 hours, cells were processed for quantitative RT-PCR analysis, in which 500 ng total RNA was used per condition. Constructs that displayed sufficient RNAi activity were used for lentivirus generation according to standard techniques (Wiznerowicz and Trono 2003 J. Virol. 77:8957-8961) (Table 2).

Table 2 shows knockdown efficiency of the different shRNA viruses.

| | Notch1 mRNA | |
|---|---|---|
| | expression | Hes1 mRNA expression |
| | (Mean ± SEM) | (Mean ± SEM) |
| | n = 3 | n = 3 |
| Le-Scrambled-DsRed | 100.0 ± 0.0 | 100.0 ± 0.0 |
| Le-shNotch1-DsRed | 19.7 ± 5.2 | 108.8 ± 5.4 |
| Le-shHes1-DsRed | 107.2 ± 4.9 | 21.8 ± 6.3 |

The data illustrated in Table 7 represent the efficiency of knockdown of the different shRNA viruses, measured by quantitative RT-PCR. All samples are corrected for RNA input (endogenous control: actin) and normalized to conditions transduced with control virus (Le-Scrambled-DsRed).

One construct of each target, shNotch1 and shHes1, was selected for further use in the RNAi experiments, generating lentivirus Le-shNotch1 and Le-shHes1, respectively. The shNotch1 target sequence is GGAAGGCUAUGACCAUGGA (SEQ ID NO:1) and shHes1 target sequence is AGAUCAACGCCAUGACCUA (SEQ ID NO:2). The selected target sequences match 100% with rat Notch1 and Hes1 mRNA sequences. Negative control RNAi construct was shScrambled (GGUAUCUACUAGAUGUACU) (SEQ ID NO:3). Primary acinar exocrine cells were infected prior to monolayer formation at MOI 50 (n=3). Transduction efficiency is presented in FIG. 8. pLVTHM Ngn3 shRNA construct and negative control pLVTHM Scrambled shRNA were designed as described previously (Baeyens et al. 2006 Cell Death Differ. 13:1892-1899). Thermostabile red-shifted Firefly Luciferase was cut out of pGex_Ppy_TS_Red (kind gift from B. Branchini) (Branchini et al. 2007 Anal. Biochem. 361:253-262) by BamHI-XhoI and subcloned in BamHI-XhoI sites of pHR′Trip-CMV-IRES-tNGFR-SIN, generating pHR′Trip-CMV-TS_FLuc_Red-IRES-tNGFR-SIN. This construct was selected for further use, generating lentivirus Le-pHR′Trip-CMV-TS_FLuc_Red-IRES-tNGFR-SIN. Control- or differentiation factor-treated acinar exocrine cells were infected prior to implantation at MOI 50 (n=4).

Immunocytochemistry

Immunocytochemical staining of the monolayers was performed directly in the 24-well plates. For this purpose, the cell monolayers were fixed for 10 min with 4% buffered formaldehyde followed by 20 min methanol (−20° C.) for cell permeabilization. Tissues were fixed with the same fixative for 4 hours and processed for paraffin embedding. Paraffin sections were used for immunostaining as described (Bouwens et al. 1994 Diabetes 43:1279-1283). Primary antibodies used in this study are polyclonal anti-insulin (C. Van Schravendijk, VUB, Brussels) (Bouwens et al. 1994 Diabetes 43:1279-1283; Bouwens and De 1996. J. Histochem. Cytochem. 44:947-951), polyclonal anti-rat C-peptide-I (O. D. Madsen, Hagedorn Research Institute, Gentofte, Denmark) (Blume et al. 1992. Mol. Endocrinol. 6:299-307), polyclonal anti-Pdx1 (O. D. Madsen) (Rooman et al. 2000 Diabetologia 43:907-914), polyclonal anti-Ngn3 (Schwitzgebel et al. 2000. Development 127:3533-3542), monoclonal anti-cytokeratin-20 (CK20) (Novocastra) (Bouwens et al. 1994 Diabetes 43:1279-1283; Bouwens and De 1996 J. Histochem. Cytochem. 44:947-951), monoclonal anti-synaptophysin (Novocastra), polyclonal anti-MafA (Bethyl Laboratories) (Nishimura et al. 2006 Dev. Biol. 293:526-539), polyclonal anti-Glut2 (Wak-Chemie), polyclonal anti-Mist1 (SF Konieczny) (Pin et al. 2000 Anat. Rec. 259:157-167), polyclonal anti-luciferase (Promega Benelux B. V.), polyclonal anti-IAPP (Advanced Chemtech), polyclonal anti-ChromograninA (Tebu-Bio), anti-BrdU (MP Biomedicals) and polyclonal anti-α-amylase (Sigma). Secondary antibodies conjugated with fluorescein isothiocyanate, tetramethylrodamine isothiocyanate or Cy5 (Jackson Immunoresearch) were used as described previously (Gu et al., 2002 Development 129:2447-2457). DNA was visualized using Hoechst 33342 (Invitrogen).

In Vitro Functionality

Cellular insulin content and insulin released in the medium were measured by radio-immunoassay (Pipeleers et al. 1985 Endocrinology 117:806-816). To study glucose-stimulated insulin release, insulin in the culture medium was measured after a 4 hour incubation in basal medium containing 2.5 mM glucose, followed by a 4 hour incubation in 20 mM glucose (serum-and glutamine-free HAM-10 medium, Gibco) (Lobner et al. 2002 Diabetes 51:2982-2988).

Microscopy

All histological images were acquired with a Nikon TE2000E inverted microscope using NIS AR2.30 Imaging Software or a Leica TCS SP confocal microscope using Volocity LE Imaging Software.

Transplantation

Eight-week old BALB/cAnNCrl-nuBR nude mice (Charles River Laboratories) weighing 18-22 g were used as recipients for transplantation. To transplant the monolayer cells, they were cultured on collagen S from calf skin (type I; Roche Molecular Biochemicals) and detached with collagenase P (Roche Molecular Biochemicals). Cell pellets (containing 300,000 cells) were implanted under the kidney capsule (Keymeulen et al. 1996 Diabetes 45:1814-1821) of nude mice that had been injected intravenously with 70 mg/kg alloxan 48 hours prior to transplantation. Blood glucose levels were monitored in samples obtained from the tail vein of fed mice by using Glucocard Memory strips (A. Menarini Diagnostics Benelux). All animal experimentation was approved by the Ethical Committee of the Free University of Brussels.

In Vivo Imaging

Mice were imaged in prone position on day 1, 4, 7, 15 and 30 after grafting and after removal of the graft-bearing kidney. Mice were anesthetized with a mixture of oxygen/isoflurane using an Inhalation Anesthesia System (VetTech solutions LTD), 5% isoflurane for induction, 2.5% isoflurane for maintenance. D-luciferin (Promega) was injected at 150 mg/kg mouse body weight via the tail vein. Immediately after D-luciferin administration, mice were imaged using the Photo Imager (Biospace). The photon emission was measured dynamically using the large field of view setting and registered using the photon counting technology (Biospace) during 600 seconds. For image analysis an elliptical region of interest (ROI) was drawn over the graft location. A background ROI was taken on the supporting table (calculation of aspecific noise), and was substracted from the specific graft signal. The area of the ROI was kept constant. For calculation of the mean graft photon emission (PEₘₑₐₙ) per group (n=4) for every timepoint mean, analyzed, the AUC of 5-second-interval counts over 600 seconds after substrate administration was calculated per individual animal. This was expressed as photons/s/steredian using the conversion factor 1 cpm=28 photons/s/steredian (ph/s/sr), provided by the camera manufacturer (Biospace). Bioluminescent pseudocolour images displayed in FIG. 6 are shown superimposed on photographic images of the mouse, with the most intense luciferase signal shown as red and the weakest signal shown as blue.

Statistical Analyses

In vitro data were analyzed using a two-tailed, paired Student t-test, in vivo data were analyzed using two-way ANOVA and statistical significance was accepted at a confidence interval <0.05 (Prism v4.0). Mean values are given±s.e.m. Number of independent experiments is indicated in the text.

### Example 2

### Inhibition of Notch Signaling Promotes the Acinar-to-Beta Cell Reprogramming

It was previously documented the transient re-expression of the pro-endocrine transcription factor Ngn3 in rat acinar cell cultures stimulated with EGF and LIF, which typically occurs in about 9% of the cells. In pancreas organogenesis the number of Ngn3⁺ endocrine precursors is limited by the lateral specification through interaction of Delta-like ligands (Dll) with the Notch1 receptor. It was examined whether re-activation of this signaling pathway might act as a limiting factor for in vitro beta cell neogenesis from adult acinar cell cultures. EGF/LIF-treated acinar cell cultures contained high levels of mRNA encoding Notch1 (FIG. 1A) and its target Hes1 (FIG. 1B), transcript levels of the ligands Jagged1 and Dll4 (FIGS. 1C and 1D) over a period of 72 h, and also a marked expression of the Hes1-inhibitor Hes6 after 24 h (FIG. 1E). Protein expression of the active intracellular domain of Notch1 (Notch1-IC) and of Hes1 was confirmed on immunoblot (FIG. 1F). The lower expression of Hes1 protein after 24 h of treatment is concordant with the observed increase in Hes6 mRNA at this timepoint. This suggests a temporary Hes6-mediated inhibition of Hes1 induced by the differentiation factors. To assess the contribution of the Notch1 pathway, recombinant activating ligands (Dll4 and Jagged1) were immobilized on the bottom of the culture plates before adding cells. Exposure to these ligands strongly inhibited expression of Ngn3 (FIG. 2A), Pdx1 (a beta cell transcription factor, FIG. 2C) and insulin, and reduced the number of newly formed beta cells by 80% (FIG. 2E). These data suggest that activation of Notch signaling reinstated lateral inhibition and restricted the beta cell neogenesis. Relief of the Notch inhibitory signal was assessed by adding an excess of Notch1 extracellular domain (Notch1-EC) protein in the culture medium. This resulted in a marked increase in the proportion of cells positive for Ngn3 (FIGS. 2A and 2B) (Table 3), Pdx1 (FIGS. 2C and 2D) and insulin (FIGS. 2E and 2F) with a 3-fold higher number of insulin-positive cells (Table 3), thus significantly improving beta cell neogenesis.

Table 3 illustrates the absolute number of insulin- and neurogenin3-positive cells after treatment of the acinar cells with different differentiation factors.

| | Number of | Total cell |
|---|---|---|
| | insulin-positive cells | number |
| Analysis after 72 h of | (Mean ± SEM) | (Mean ± SEM) |
| treatment | n = 7 | n = 7 |
| Untreated controls | 20.71 ± 4.35 | 4071.43 ± 94.83 |
| EGF + LIF treated | 469.86 ± 15.16 | 4887.57 ± 100.15 |
| EGF + LIF + Notch1-EC treated | 1579.00 ± 57.04 | 4952.71 ± 149.39 |

| | Number of | |
|---|---|---|
| | neurogenin3- | Total cell |
| | positive cells | number |
| Analysis after 48 h of | (Mean ± SEM) | (Mean ± SEM) |
| treatment | n = 5 | n = 5 |
| Untreated controls | 3.46 ± 0.68 | 4430.00 ± 180.86 |
| EGF + LIF treated | 489.33 ± 34.76 | 5002.05 ± 237.36 |
| EGF + LIF + Notch1-EC treated | 1461.67 ± 75.46 | 5112.67 ± 84.13 |

The data illustrated in Table 3 represent the primary analysis of the absolute number of insulin- and neurogenin-positive cells in the different conditions tested. Epidermal growth factor (EGF, 10 ng/ml); Leukemia Inhibitory Factor (LIF, 40 ng/ml); Extracellular part of Notch1 receptor (Notch1-EC, 10 mg/ml).

The observed effect could be explained by Notch1-EC acting as a competitive inhibitor and reducing the interaction of endogenous ligands on cultured cells with the Notch receptor. To further dissect the underlying mechanism, the effects of virally delivered shRNAs directed against either Notch1 or its effector Hes1 were evaluated. The efficiency of transduction did not differ between the various conditions, with on average about 25% of the cells expressing the reporter (FIG. 8). Silencing the receptor or its downstream target rendered the cells insensitive both to endogenous Notch signaling and recombinant ligand induced signaling, and resulted in an increased proportion of the acinar cells adopting a beta cell phenotype compared to control shRNA-treated conditions (FIG. 3A-E). The effect of silencing the Notch pathway was most pronounced when cells had been previously exposed to Notch ligands. More than 75% of the transduced cells adopted a beta cell phenotype in the latter condition (FIGS. 3F and 3G) (Tables 4 and 5). The role of Ngn3 as key regulator of the acino-insular conversion in cells treated with EGF, LIF and Notch1-EC was confirmed using shRNA directed against Ngn3 (FIG. 3H-3J). Cells with a stable silencing of Ngn3 mRNA were unable to respond to the given treatment, resulting in a downregulation of the beta cell number (Table 6). These findings confirm our hypothesis that Notch signaling acts as an inhibitor of induced beta cell differentiation in primary acinar cell cultures.

Table 4 shows RNAi mediated inhibition of Notch1 in acinar differentiation to beta cells.

| | Number of | Number of | Number of | |
|---|---|---|---|---|
| | insulin-positive | DsRed-positive | insulin/DsRed- | Total cell |
| | cells | cells | positive cells | number |
| | (Mean ± SEM) | (Mean ± SEM) | (Mean ± SEM) | (Mean ± SEM) |
| | n = 3 | n = 3 | n = 3 | n = 3 |
| EGF + LIF | 575.00 ± 12.34 | 1384.00 ± 66.53 | 130.67 ± 3.84 | 5753.67 ± 96.07 |
| treated | | | | |
| Le-Scrambled | | | | |
| EGF + LIF | 1234.00 ± 21.96 | 1447.33 ± 33.33 | 493.00 ± 2.08 | 5895.00 ± 63.54 |
| treated | | | | |
| Le-shNotch1 | | | | |
| EGF + LIF + | 109.00 ± 8.96 | 1148.67 ± 27.67 | 26.00 ± 1.53 | 4995.67 ± 181.67 |
| Jagged1 treated | | | | |
| Le-Scrambled | | | | |
| EGF + LIF + | 1030.67 ± 32.42 | 1243.00 ± 42.22 | 1011.33 ± 30.94 | 5330.33 ± 220.53 |
| Jagged1 treated | | | | |
| Le-shNotch1 | | | | |
| EGF + LIF + DII4 | 165.33 ± 4.91 | 1239.33 ± 64.33 | 39.66 ± 1.20 | 5061.33 ± 139.93 |
| treated | | | | |
| Le-Scrambled | | | | |
| EGF + LIF + DII4 | 1034.00 ± 69.51 | 1322.00 ± 75.08 | 1004.00 ± 65.68 | 5667.33 ± 184.76 |
| treated | | | | |
| Le-shNotch1 | | | | |
| EGF + LIF + | 1758.33 ± 61.60 | 1309.67 ± 43.21 | 409.33 ± 21.40 | 5600.67 ± 172.85 |
| Notch1-EC treated | | | | |
| Le-Scrambled | | | | |
| EGF + LIF + | 2072.67 ± 29.17 | 1396.33 ± 55.57 | 607.33 ± 9.35 | 5969.00 ± 82.35 |
| Notch1-EC treated | | | | |
| Le-shNotch1 | | | | |

The data shown in Table 4 represent the primary analysis of the absolute number of insulin-positive cells after transduction with control virus (Le-Scrambled) or specific Notch1 silencing virus (Le-shNotch1) in the different conditions tested. Epidermal growth factor (EGF, 10 ng/ml); Leukemia Inhibitory Factor (LIF, 40 ng/ml); Extracellular part of Notch1 receptor (Notch1-EC, 10 mg/ml); Jagged1 and Dll4 (Notch ligands).

Table 5 shows RNAi mediated inhibition of Hes1 in acinar differentiation to beta cells

| | Number of | Number of | Number of | |
|---|---|---|---|---|
| | insulin-positive | DsRed-positive | insulin/DsRed- | Total cell |
| | cells | cells | positive cells | number |
| | (Mean ± SEM) | (Mean ± SEM) | (Mean ± SEM) | (Mean ± SEM) |
| | n = 3 | n = 3 | n = 3 | n = 3 |
| EGF + LIF treated | 618.33 ± 24.74 | 1347.67 ± 47.68 | 140.00 ± 9.02 | 5962.00 ± 52.79 |
| Le-Scrambled | | | | |
| EGF + LIF treated | 1235.00 ± 112.37 | 1554.67 ± 74.73 | 493.00 ± 37.07 | 6078.67 ± 171.20 |
| Le-shHes1 | | | | |
| EGF + LIF + Jagged1 | 116.00 ± 8.72 | 1174.00 ± 43.96 | 25.67 ± 3.48 | 5140.33 ± 89.19 |
| treated | | | | |
| Le-Scrambled | | | | |
| EGF + LIF + Jagged1 | 1078.33 ± 123.14 | 1501.33 ± 19.89 | 1020.33 ± 127.57 | 5809.00 ± 160.07 |
| treated | | | | |
| Le-shHes1 | | | | |
| EGF + LIF + DII4 treated | 155.33 ± 12.47 | 1198.33 ± 85.60 | 34.67 ± 2.18 | 5191.33 ± 265.02 |
| Le-Scrambled | | | | |
| EGF + LIF + DII4 treated | 1043.33 ± 21.67 | 1554.00 ± 24.79 | 988.67 ± 12.34 | 6001.67 ± 104.32 |
| Le-shHes1 | | | | |
| EGF + LIF + Notch1-EC | 1941.00 ± 73.57 | 1309.67 ± 43.21 | 402.33 ± 25.11 | 6231.33 ± 97.40 |
| treated | | | | |
| Le-Scrambled | | | | |
| EGF + LIF + Notch1-EC | 2131.67 ± 60.36 | 1463.00 ± 97.18 | 599.33 ± 8.45 | 6179.00 ± 109.16 |
| treated | | | | |
| Le-shHes1 | | | | |

The data shown in table 5 represent the primary analysis of the absolute number of insulin-positive cells after transduction with control virus (Le-Scrambled) or specific Hes1 silencing virus (Le-shHes1) in the different conditions tested. Epidermal growth factor (EGF, 10 ng/ml); Leukemia Inhibitory Factor (LIF, 40 ng/ml); Extracellular part of Notch1 receptor (Notch1-EC, 10 mg/ml); Jagged1 and Dll4 (Notch ligands).

Table 6 shows RNAi mediated inhibition of Ngn3.

| | Number of | Number of | Number of | |
|---|---|---|---|---|
| | insulin-positive | eGFP-positive | insulin/eGFP- | Total cell |
| Analysis | cells | cells | positive cells | number |
| after 72 h | (Mean ± SEM) | (Mean ± SEM) | (Mean ± SEM) | (Mean ± SEM) |
| of treatment | n = 3 | n = 3 | n = 3 | n = 3 |
| EGF + LIF | 1574.00 ± 26.54 | 1189.33 ± 33.71 | 368.33 ± 23.38 | 4929.67 ± 88.72 |
| treated | | | | |
| Le-Scrambled | | | | |
| EGF + LIF | 1162.33 ± 32.99 | 1142.67 ± 57.49 | 6.00 ± 1.53 | 5065.33 ± 105.92 |
| treated | | | | |
| Le-shNgn3 | | | | |

The data shown in table 6 represent the primary analysis of the absolute number of insulin-positive cells after transduction with control virus (Le-Scrambled) or specific Ngn3 silencing virus (Le-shNgn3) after treatment with EGF and LIF. Epidermal growth factor (EGF, 10 ng/ml); Leukemia Inhibitory Factor (LIF, 40 ng/ml).

### Example 3

### The Newly Formed Beta Cells are of Acinar Origin

Combined EGF, LIF and Notch1-EC treatment resulted in 30% of the cells adopting a beta cell phenotype, compared to 0.5% in control conditions (FIG. 2). However, the acinar origin of these cells still needs to be demonstrated. To achieve this goal we set up a non-genetic lineage tracing system based on the use of fluorescent lectins. Fluorescent Wheat Germ Agglutinin (WGA), that binds to N-acetyl glucosamine, exclusively expressed on acinar cells in the pancreas, was micro-injected directly into the pancreatic parenchyma at multiple sites. Lectin binding and internalization was allowed for 72 h and animals were sacrificed for microscopical analysis. WGA fluorescence was found in the cytoplasm of acinar cells only and was never observed in other cell types like centroacinar, duct or islet cells (FIGS. 4A and 4B, FIG. 9A). Collagenase digestion of the pancreas and subsequent cell isolation confirmed the WGA specificity as about 60% of the isolated acinar cells displayed strong lectin positivity, whereas none of the other cell types contained the fluorescent WGA (FIGS. 4C and 4D, FIG. 9B-D). When the labeled acinar cells were treated in vitro with EGF, LIF and Notch1-EC to induce acino-insular conversion, approximately 60% of the insulin-positive cells displayed cytoplasmic WGA positivity at the end of the culture (FIGS. 4E and 4F). This confirms that acinar cells can be stimulated to undergo a phenotypical switch towards beta cells under the present culture conditions. The contribution of proliferation to this process was limited, as only 0.3±0.1% of the Ngn3-positive and 0.5±0.2% of the insulin-positive cells incorporated BrdU after a 6 h labeling pulse (n=3). Redistribution of the WGA lectin in vitro from dying acinar cells to few contaminating islet cells should however be excluded. Therefore WGA labeling was also performed in vitro at the moment when pro-endocrine specification was already initiated, i.e. when Ngn3 starts to appear. Subsequently, WGA label was found only in non-endocrine cells. No lectin label could be found in Ngn3-positive cells or in insulin-positive cells at 3 h and 24 h after labeling (Table 7). This observation excludes the possibility of in vitro uptake of released WGA by cells already committed to an endocrine fate.

Table 7 shows a comparison of in vivo and in vitro WGA lectin labeling.

| | Neurogenin3-positive | Insulin-positive |
|---|---|---|
| | cells | cells |
| | (Mean ± SEM) | (Mean ± SEM) |
| % WGA labeled cells | n = 4 | n = 4 |
| In vivo labeling | 60.82 ± 2.68 | 62.07 ± 1.72 |
| In vitro labeling | 0.32 ± 0.24 | 0.19 ± 0.16 |
| (after 48 h pro-endocrine | | |
| treatment) | | |

The data represented in Table 7 represent the proportion of neurogenin3- and insulin-positive cells positive for the lectin label after treatment with EGF, LIF and Notch1-EC. Epidermal growth factor (EGF, 10 ng/ml); Leukemia Inhibitory Factor (LIF, 40 ng/ml); Extracellular part of Notch1 receptor (Notch1-EC, 10 mg/ml).

To further exclude the in vitro contribution of pre-existing beta cells to the observed neogenesis of insulin-positive cells, islet beta cells were labeled in vitro using Concanavalin A (ConA) (Maylie-Pfenninger and Jamieson 1979, J. Cell Biol. 80:77-95); these cells were then mixed with the WGA labeled acinar cells at the start of the culture. As in the other experiments, the cells had been treated with alloxan to selectively destroy contaminating beta cells before the start of the pro-endocrine treatment. After beta cell induction treatment, only WGA lectin could be found in the newly formed beta cells. The absence of ConA at the end of the culture demonstrates that no contaminating beta cells escaped the alloxan-toxicity and thus did not contribute to the observed neogenesis.

### Example 4

### Newly Formed Beta Cells are Relatively Immature

To evaluate the maturity of the new beta cells, they were immunostained for typical beta cell markers. 97% of the insulin-expressing cells co-expressed the beta cell markers Pdx1, C-peptide and synaptophysin. However, only about 25-30% of these cells expressed the other mature beta cell markers MafA, Glut2, IAPP and Chromogranin A, suggesting their immature nature (FIG. 5A). Although the initial cellular insulin content did not differ between new and islet beta cells (respectively 26.1±0.9 pg and 28.0±0.9 pg, n=4, p=0.14) (FIG. 5B), glucose-stimulated insulin secretion by the newly formed beta cells was 50% of islet beta cell capacity (under identical conditions) (FIG. 5C). These findings emphasize the immature nature of the acinar-derived beta cells.

### Example 5

### New Beta Cells Mature In Vivo and Restore Normoglycemia

Function of the new beta cells was further examined after transplantation of the cells in diabetic immuno-deficient mice. A graft of 300,000 cells containing 100,000 insulin-positive cells was able to restore normoglycemia in initially hyperglycemic recipient mice (blood glycemia above 300 mg/dl) within 4 days (FIG. 6A). Blood glycemia remained stable and rapidly reverted to hyperglycemia when the engrafted kidney was removed, demonstrating the graft was directly responsible for blood glucose control (FIG. 6A). Animals implanted with control grafts (acinar-derived cells without beta cell induction treatment) failed to restore normoglycemia at all time points (FIG. 6A). The mean body weight of the animals did not differ between the two groups at all time points (FIG. 3A). Graft survival was monitored via bioluminescent in vivo imaging. Grafted cells were transduced with Lenti-pCMV-Luciferase (Thermostable red-shifted Firefly Luciferase (Branchini et al. 2007, Anal. Biochem. 361:253-262) 48 h before implantation under the kidney capsule. Luciferase expression of both control grafts and grafts of cells that had been treated with EGF, LIF and Notch1-EC did not differ significantly prior to implantation (FIG. 10B). This non-invasive real-time analysis revealed a stable luciferase activity and thus no marked increase in graft size following beta cell induction treatment (FIGS. 6B and 6C), with the modest increase in signal intensity related to the improved vascularisation of the graft. In contrast, control grafts rapidly decreased in luciferase activity, indicating a profound loss in graft size (FIG. 6B). The latter was confirmed by histological examination. Histological analyses of the graft showed minimal presence of alpha cells (FIG. 11A), as well as further maturation of the insulin-positive cells since the majority of these cells now did co-express the functional beta cells markers Glut2 (FIG. 7C), MafA (FIG. 7D), IAPP and chromogranin A (FIG. 11B-C) while remaining to express Pdx1 (FIG. 7A), C-peptide (FIG. 7B) and synaptophysin (FIG. 11D). Histological analysis showed co-expression of insulin and luciferase in these grafts (FIG. 7E). No sign of endogenous beta cell recovery in the pancreas was found (FIG. 7F). Thus, acinar-derived beta cells are capable to form stable grafts which can restore normoglycemia in diabetic recipients.

### Example 6

### New Beta Cells Mature In Vivo and Restore Normoglycemia

Exocrine acinar cells can be converted into endocrine beta cells in vitro by stimulation with physiological signals and interfering with Notch signaling. The Aplicant has shown that not only endocrine genes are induced, but also Notch related genes are markedly upregulated after stimulation of cells with EGF and LIF treatment. In the present model, exposure of the cells to the Notch1 ligands Dll4 and Jagged1 enhances the inhibitory effect of endogenous Notch signaling on the acino-insular conversion, resulting in a near complete abrogation of the beta cell neogenesis. RNAi mediated silencing of the pathway, by knock-down of Notch1 or its effector Hes1, increased the acinar-to-beta cell conversion induced by the differentiation factors. Blocking Notch signaling by exposing the cells to excess amounts of Notch1-EC mimics the latter effect, and maximizes neogenesis with more than 30% of acinar cells that adopt a beta cell phenotype. These observations indicate the Notch pathway as a major obstacle to overcome when attempting to convert acinar cells into beta cells.

With the specific method of cell tracing using lectins an alternative for genetic lineage tracing is offered, since transfection/transduction difficulties or lack of available transgenic animals can be a major obstacle for the latter approach. Lectin-based cell labeling was shown to be stable, specific and reached an efficiency of 62%; it allowed follow-up of cell cultures for at least 10 days. Using this technique, the acinar origin of the insulin-positive cells emerging in the monolayer culture was unequivocally shown. Previous papers have used genetic lineage tracing (with lower labeling efficiency) to demonstrate the ability of mouse acinar cells to undergo a phenotypical switch towards beta cells. The present model represents a major improvement of previous protocols as for the first time the possibility is revealed for large scale acinar-to-beta cell conversion using physiological conditions without need for genetic modification. It was also shown that newly formed beta cells mature after transplantation at an ectopic site and can be used to re-establish normoglycemic control.

Adult rat acinar tissue in vitro can be considered as a valid source of functional beta cells, and thus may offer perspectives for increasing the transplantable beta cell pool. Conversions between mature cell types may also find applications in other fields of replacement therapy.

Summarised, exocrine pancreatic cells such as acinar cells in the mammalian pancreas are highly differentiated cells which, however, retain a remarkable degree of plasticity. In accordance with the present invention a culture model was developed to convert pancreatic cells, and preferably acinar cells into endocrine beta cells based on reprogramming dedifferentiated pancreatic cells and preferably acinar cells with physiological factors, EGF and LIF. A lectin-based cell labeling method was used to demonstrate the acinar origin of newly formed insulin-expressing beta cells. The phenotypic conversion is controlled by Notch signaling, which is known to control cell differentiation in embryonic pancreas. Physiological or RNAi-based interference with this signaling allows modulating the acinar cell susceptibility to the differentiation-inducing factors and significantly improves beta cell neoformation. The newly formed beta cells further mature when transplanted ectopically, and are capable of restoring normal blood glycemia in diabetic recipients. This efficient way to generate beta cells by adult cell type conversion has application in cell replacement therapy of for instance type-1 or type-2 diabetes.

1. An in vitro method for generating insulin-producing beta cells from a population of mammalian cells comprising dedifferentiated exocrine pancreatic cells comprising the step of culturing said dedifferentiated exocrine pancreatic cells in a culture medium in the presence of:
at least one agent that is able to inhibit the Notch1 signaling pathway in said dedifferentiated exocrine pancreatic cells, and
at least one ligand of the gp130 receptor and/or at least one ligand of the EGF receptor.

2. Method according to claim 1, wherein said dedifferentiated exocrine pancreatic cells are cultured in a culture medium in the presence of at least one agent that is able to inhibit the Notch1 signaling pathway in said dedifferentiated exocrine pancreatic cells, at least one ligand of the gp130 receptor and at least one ligand of the EGF receptor.

3. Method according to claim 1, wherein the agent that is able to inhibit the Notch1 signaling pathway is an agent capable of causing RNA interference with Notch1 or a Hes gene.

4. Method according to claim 1, wherein the agent that is able to inhibit the Notch1 signaling pathway is an RNA interfering agent selected from the group consisting of short interfering nucleic acid (siNA), short interfering RNA (siRNA), double-stranded RNA (dsRNA), micro-RNA (miRNA), and short hairpin RNA (shRNA).

5. (canceled)

6. Method according to claim 4, wherein said RNA interfering agent is a shRNA having at least 95% sequence identity with Notch1 mRNA.

7. Method according to claim 6, wherein said RNA interfering agent is a shRNA having a sequence as represented in SEQ ID NO:1.

8. Method according to claim 4, wherein said RNA interfering agent is a shRNA having at least 95% sequence identity with the mRNA of a Hes gene.

9. Method according to claim 8, wherein said RNA interfering agent is a shRNA having a sequence as represented in SEQ ID NO:2

10. Method according to claim 1, wherein said agent that is able to inhibit the Notch1 signaling pathway is a Notch1-inhibiting agent.

11. Method according to claim 10, wherein said Notch1-inhibiting agent is Notch1-EC.

12. Method according to claim 1, wherein said ligand of said gp130 receptor is LIF.

13. Method according to claim 1, wherein said ligand of said EGF receptor is EGF.

14. (canceled)

15. (canceled)

16. The method according to claim 1, wherein said dedifferentiated exocrine pancreatic cells are dedifferentiated exocrine acinar cells.

17. A population of mammalian pancreatic cells comprising more than 20% of insulin-positive cells obtainable by the method of claim 1.

18. Population of mammalian pancreatic cells according to claim 17, wherein said insulin-producing beta cells are generated from dedifferentiated exocrine pancreatic cells, preferably from exocrine acinar cells.

19. Population of mammalian pancreatic cells according to claim 17, wherein said cell population after exposure to 20 mM glucose for 2 hours at 37° C. in HamF10 medium secretes at least half of the amount of insulin that is secreted by endogenous beta cells under identical conditions.

20-22. (canceled)

23. A pharmaceutical composition comprising a therapeutically effective amount of a population of mammalian pancreatic cells according to claim 17 and at least one pharmaceutically acceptable carrier.

24-25. (canceled)

26. A method of treating a patient suffering from type 1 or type 2 diabetes comprising administering to said patient a population of mammalian pancreatic cells according to claim 17.


SEQUENCE LISTING (15 sequences)

SEQ ID NO: 1 - RNA, 19 nt, Artificial sequence
  ShNotch1 target sequence
ggaaggcuaugaccaugga

SEQ ID NO: 2 - RNA, 19 nt, Artificial sequence
  shHes1 target sequence
agaucaacgccaugaccua

SEQ ID NO: 3 - RNA, 19 nt, Artificial sequence
  scrambled sequence
gguaucuacuagauguacu

SEQ ID NO: 4 - DNA, 22 bp, Artificial sequence
  primer
cacaccaacgtggtcttcaagc

SEQ ID NO: 5 - DNA, 22 bp, Artificial sequence
  Primer
tagacaatggagccacggatgt

SEQ ID NO: 6 - DNA, 20 bp, Artificial sequence
  primer
ttcagcgagtgcatgaacga

SEQ ID NO: 7 - DNA, 21 bp, Artificial sequence
  Primer
taggtcatggcgttgatctgg

SEQ ID NO: 8 - DNA, 21 bp, Artificial sequence
  Primer
accacctgcgaagtgattgac

SEQ ID NO: 9 - DNA, 19 bp, Artificial sequence
  Primer
tgaatttgcctcccgactg

SEQ ID NO: 10 - DNA, 21 bp, Artificial sequence
  Primer
ggcaaactgcagaaccacaca

SEQ ID NO: 11 - DNA, 21 bp, Artificial sequence
  Primer
gacatgccggcttttcactgt

SEQ ID NO: 12 - DNA, 20 bp, Artificial sequence
  Primer
gctgccatccttaagtttgc

SEQ ID NO: 13 - DNA, 21 bp, Artificial sequence
  Primer
agagccaccaatccacacaga

SEQ ID NO: 14 - DNA, 21 bp, Artificial sequence
  Primer
actatcggcaatgagcggttc

SEQ ID NO: 15 - DNA, 22 bp, Artificial sequence
  primer
ggtcatcagggttaccatacaa